(19) United States
(12) Patent Application Publication
(10) Pub. No.: US 2019/0227072 A1
(43) Pub. Date: Jul. 25, 2019

(54) Biomarkers for Carcinogenesis and Uses Thereof

(71) Applicant: GEORGETOWN UNIVERSITY, Washington, DC (US)

(72) Inventors: Fung-Lung Chung, Rockville, MD (US); Ying Fu, Potomac, MD (US); Aiwu Ruth He, Falls Church, VA (US)

(73) Assignee: GEORGETOWN UNIVERSITY, Washington, DC (US)

(21) Appl. No.: 16/324,797

(22) PCT Filed: Aug. 11, 2017
(86) PCT No.: PCT/US2017/046531

§ 371 Date: Feb. 11, 2019

Related U.S. Application Data
(60) Provisional application No. 62/374,255, filed on Aug. 12, 2016.

(51) Int. Cl. G01N 33/574; A61K 31/353; C12Q 1/02; A61K 31/385

(52) U.S. Cl. CPC G01N 33/57488; A61K 31/385; C12Q 1/025; A61K 31/353

(57) ABSTRACT

Provided herein are methods of reducing the recurrence of liver cancer in a subject.

This application claims priority to U.S. Provisional Application No. 62/374,255, filed Aug. 12, 2016, which is hereby incorporated in its entirety by this reference.

### STATEMENT REGARDING FEDERALLY FUNDED RESEARCH

This invention was made with government support under Grant No. RO1-CA-134892 awarded by the National Cancer Institute. The government has certain rights in the invention.

### BACKGROUND

Liver cancer is the third leading cause of cancer-related deaths worldwide. Often, by the time liver cancer is diagnosed, treatment options are limited. Surgical removal of the malignant tissue is generally an option, but, for some, a liver transplant is the only viable hope. No effective biomarkers are available to detect liver cancer or to identify subjects at risk of a recurrence of liver cancer.

### SUMMARY

Provided herein are methods for reducing the recurrence of liver cancer in a subject. The methods include the steps of (a) obtaining a hepatic tissue sample from a subject; (b) treating the subject for liver cancer; (c) determining a level of γ-hydroxy-1, N2-propanodeoxyguanosine (γ-OHPdG) in the sample; (d) comparing the detected level of step (c) to one or more control values. An elevated level of γ-OHPdG indicates the subject is at risk for the recurrence of liver cancer. An antioxidant is then administered to the subject at risk for recurrence.

Also provided are methods that include the steps of (a) obtaining a hepatic tissue sample from a subject; (b) treating the subject for liver cancer; (c) determining a level of 7-(1′,2′-dihydroxyheptyl)-1,N⁶-ethenodeoxyadenosine (DHHεdA) in the sample; (d) comparing the detected level of step (c) to one or more control values. An elevated level of DHHεdA indicates the subject is at risk for the recurrence of liver cancer. An antioxidant is then administered to the subject at risk for recurrence.

### DESCRIPTION OF THE FIGURES

FIG. 1 shows representative genotyping results of wildtype (WT) and xeroderma pigmentosum group A correcting knockout (XPA (−/−)) mice.

FIG. 2A-E shows the results of a 72-week tumor bioassay of WT and Xpa^{-/-} mice. FIG. 2A shows representative livers of WT and XPA^{-/-} mice at 72 weeks after feeding a control diet. FIG. 2B shows tumor multiplicity (top panel), maximum tumor diameter (middle panel) and incidences (bottom panel). FIG. 2C shows γ-OHPdG levels detected by LC-MS/MS in livers of mice at 8-week, 16-week and 32-week time points. FIG. 2D shows GSH/GSSG ratios in livers of mice. FIG. 2E shows average body weights of WT and XPA^{-/-} mice at 72 weeks. n=6-7 for each group. Error bars represent SD values. *p<0.05.

FIG. 3 shows hematoxylin and eosin (H&E) staining of WT and XPA−/− mice livers. The scores on the graph are as follows: no steatosis=0, minimum steatosis=1, mild steatosis=2, moderate steatosis=3, severe steatosis=4.

FIG. 4A-D shows γ-OHPdG levels detected by the LC-MS/MS method in C3H mouse tissues at 8-week, 16-week and 32-week time points. FIGS. 4A and 4B provide a comparison in liver (FIG. 4A) and lung (FIG. 4B) between male and female XPA^{-/-} mice fed with the same control diet. FIGS. 4C and 4D show a comparison between WT and XPA^{-/-} mice in lung (FIG. 4C) and liver (FIG. 4D). n=3-7 for each group. Error bars represent SD values. * p<0.05.

FIG. 5 shows the levels of γ-OHPdG in the liver of Long-Evans (LE) and Long-Evans Cinnamon (LEC) rats detected by LC-MS/MS. N=12 rats. *p<0.05, ***p<0.001.

FIG. 6A-C shows profiles of mutations found in liver tumors from two XPA^{-/-} mice. FIG. 6A shows the distribution of somatic mutations in each tumor sample. FIG. 6B shows the frequency of indels and nucleotide substitutions in each tumor sample. FIG. 6C shows the percentage of tolerated and deleterious mutations (SIFT data).

FIG. 7A-C shows a 32-week bioassay of XPA^{-/-}- mice with and without the treatment of antioxidants. FIG. 7A is a diagram of the experimental protocol. FIG. 7B shows γ-OHPdG levels in the livers of XPA(−/−) mouse detected by LC-MS/MS after feeding various antioxidant diets (Theaphenon E (20 g/kg), α-lipoic acid (2 g/kg), and vitamin E (1.8 g/kg)) and a control diet. FIG. 7C shows GSH/GSSG ratio changes in the livers of mice fed different diets for 32 weeks. *p<0.05, **p<0.01. FIGS. 7D-7F show γ-OHPdG levels detected by LC-MS/MS in livers and lungs from mice after feeding different antioxidant diets for 8, 16, and 32 weeks, respectively. Error bars represent SD values.

FIG. 8A-E shows the results of a 72-week tumor bioassay of XPA^{-/-} mice. FIG. 8A shows representative livers of mice kept on control or different antioxidant diets (Theaphenon E, α-lipoic acid, and vitamin E) from week 4 to week 72. FIG. 8B shows tumor multiplicity. FIG. 8C shows tumor size. FIG. 8D shows tumor incidence. n=17-18 for each group. FIG. 8E shows that malondialdehyde (MDA) levels change in the livers of 32-week mice, n=6 for each group. Error bars represent SD values. *p<0.05, **p<0.01, ***p<0.001, ns=non-significant.

FIG. 9A-D shows a comparison between male and female XPA^{-/-} mice in (FIG. 9A) tumor multiplicity, (FIG. 9B) maximum tumor diameter, (FIG. 9C) incidences, and (FIG. 9D) liver γ-OHPdG levels at 32-weeks. n=3-7 for each group, error bars represent SD values.

FIG. 10A shows body weight of XPA^{-/-} mice during the 72-week period under different diets. FIG. 10B shows age-dependent changes of GSH/GSSG ratio in the livers of mice under different diets. FIG. 10C shows food consumption of mice under different diets at 12- and 13-weeks.

FIG. 11A-C shows the results of a 32-week bioassay of diethylnitrosamine- (DEN-) treated C56B/6 mice. FIG. 11A shows H&E immunohistochemistry staining of livers from mice of different age. FIG. 11B shows γ-OHPdG levels detected by LC-MS/MS in mouse livers and lungs. FIG. 11C shows that steatosis was evaluated and quantified using this scoring system: no steatosis=0, minimum steatosis=1, mild steatosis=2, moderate steatosis=3, severe steatosis=4. *p<0.05.

FIG. 12A-D shows the results of a 32-week tumor bioassay of DEN-treated C56B/6 mice fed diets with and without Theaphenon E. FIG. 12A is s diagram of an experimental protocol. 14-day old C56BL/6 male pups were given a single i.p. injection of DEN (5 mg/kg). Recipients were sacrificed 8 months later for liver tumor analysis. FIG. 12B shows representative livers of mice under a control diet and a Theaphenon E diet. FIG. 12C shows representative immunohistochemistry staining of H&E for mouse liver. FIG. 12D shows maximum tumor diameter, tumor multiplicity, and incidences. n=5 for each group. Error bars represent SD values. *p<0.05.

FIG. 13A-B shows that levels of γ-OHPdG in tumor and non-tumor regions of animal and human HCC samples showed no correlation between the tumor and the adjacent normal tissue. FIG. 13A shows levels of γ-OHPdG in tumor and non-tumor regions detected by LC-MS/MS. FIG. 13B shows levels of γ-OHPdG in human hepatocellular carcinoma (HCC) samples detected by immunohistochemistry (IHC). Samples showing the same IHC scores are overlapped in FIG. 13B.

FIG. 14A-E shows γ-OHPdG as a biomarker of survival and recurrence-free survival in HCC patients. FIG. 14A shows representative images of γ-OHPdG staining in human liver tissue from 90 HCC patients: semi-quantitative scores were calculated by adding intensity and distribution of staining: 0, 1, 2, 3 correspond to negative, weak, moderate, and intense staining and 0, 1, 2, 3 correspond to negative, focal, regional, and diffuse distribution.

FIG. 14B is a Kaplan-Meier Curve for γ-OHPdG staining that demonstrates decreased survival in patients with high γ-OHPdG tumor (p<0.0001). FIG. 14C shows representative γ-OHPdG staining of liver samples from patients at Georgetown University clinical trial. FIG. 14D is a Kaplan-Meier Curve for γ-OHPdG staining using median cut-off point (high >3, low <3) showing decreased recurrence-free survival in patients with high γ-OHPdG (bottom line) in tumors compared to patients with low γ-OHPdG in tumors (upper line) (p=0.007). FIG. 14E shows comparisons of the immunostaining score of γ-OHPdG between primary tumor and recurrent tumor in HCC patients.

FIG. 15 is a scheme showing the formation of selected cyclic DNA adducts derived from enals as secondary products of oxidized PUFAs via hydroperoxy fatty acids (FAOOH): α-OH-1,N²-propano-2′-deoxyguanosine (α-OHPdG), γ-OH-1,N²-propano-2′-deoxyguanosine (γ-OHPdG), 1,N⁶-etheno-2′-deoxyadenosine (EdA), 7-(1′,2′-dihydroxyheptyl)-1,N⁶-etheno-2′-deoxyadenosine (DHHεdA), and 1,N²-deoxyguanosine adducts of (E)-trans-4-hydroxy-2-nonenal (HNE-dG); EH-2,3-epoxy-4-hydroxynonanal, HNE—(E)-4-hydroxy-2-nonenal.

FIG. 16 is a scheme showing synthesis of immunogens. Conditions for reaction a were (2,2,6,6-tetramethylpiperidin-1-yl)oxyl (TEMPO), (diacetoxyiodo)benzene (BIB), 1:1 acetonitrole (ACN):water, 3 h. Conditions for reaction b were 80% formic acid, room temperature, 43 h. Conditions for reaction c were HNE in tetrahydrofuran (THF), 30% H₂O₂ and ACN mixed with 3 dissolved in 2:1 THF:100 mM phosphate buffer, 50° C., 7 days. Conditions for reaction d were 1-(3-(dimethylamino)propyl)-3-ethylcarbodiimide hydrochloride (EDAC), BSA or keyhole limpet hemocyanin (KLH), 0.1 M 2-(N-morpholino)ethanesulfonic acid (MES) buffer. Step e shows raising mAbs against DHHεdA by immunization of mice with antigen 5.

FIG. 17 shows the reactivity of mAbs against DHHεA-BSA by ELISA. DHHεA-BSA conjugate (×2, 100 ng) was immobilized on a plate and incubated with varying dilutions of mAbs from the six monoclonal cell lines.

FIG. 18A shows the reactivity of mAbs against normal nucleotides by competitive ELISA for dG. DHHεA-BSA conjugate (×2, 100 ng) was immobilized on a plate and incubated with mAbs and varying concentrations (0, 0.1, 1, 10 and 100 ng) of competing agents None of the six mAbs tested binds to normal nucleotides. FIG. 18B shows the reactivity of mAbs against normal nucleotides by competitive ELISA for dA. DHHεA-BSA conjugate (×2, 100 ng) was immobilized on a plate and incubated with mAbs and varying concentrations (0, 0.1, 1, 10 and 100 ng) of competing agents. None of the six mAbs tested binds to normal nucleotides. FIG. 18C shows the reactivity of mAbs against normal nucleotides by competitive ELISA for dC. DHHεA-BSA conjugate (×2, 100 ng) was immobilized on a plate and incubated with mAbs and varying concentrations (0, 0.1, 1, 10 and 100 ng) of competing agents. None of the six mAbs tested binds to normal nucleotides. FIG. 18D shows the reactivity of mAbs against normal nucleotides by competitive ELISA for T. DHHεA-BSA conjugate (×2, 100 ng) was immobilized on a plate and incubated with mAbs and varying concentrations (0, 0.1, 1, 10 and 100 ng) of competing agents None of the six mAbs tested binds to normal nucleotides.

FIG. 19A shows the reactivity of mAbs against Acr-dG (acrolein derived cyclic 1,N²-propanodeoxyguanosine adducts (OHPdG)), a commonly detected DNA adduct, by competitive ELISA. DHHεA-BSA conjugate (×2, 100 ng) was immobilized on a plate and incubated with mAbs and varying concentrations (0, 0.1, 1, 10 and 100 ng) of competing agents. None of the six mAbs tested bound to Acr-dG. FIG. 19B shows the reactivity of mAbs against 8-oxo-dG, a commonly detected DNA adduct, by competitive ELISA. DHHεA-BSA conjugate (×2, 100 ng) was immobilized on a plate and incubated with mAbs and varying concentrations (0, 0.1, 1, 10 and 100 ng) of competing agents. None of the six mAbs tested bound to 8-oxo-dG. FIG. 19C shows the reactivity of mAbs against HNE-dG, a commonly detected DNA adduct, by competitive ELISA. DHHεA-BSA conjugate (×2, 100 ng) was immobilized on a plate and incubated with mAbs and varying concentrations (0, 0.1, 1, 10 and 100 ng) of competing agents. All six mAbs tested showed moderate binding affinity to HND-dG. FIG. 19D shows the reactivity of mAbs against edA, a commonly detected DNA adduct, by competitive ELISA. DHHεA-BSA conjugate (×2, 100 ng) was immobilized on a plate and incubated with mAbs and varying concentrations (0, 0.1, 1, 10 and 100 ng) of competing agents. Moderate affinity to edA was found for two mAbs.

FIG. 20A shows the reactivity of purified 3C9C9 mAb against normal nucleotides. DHHεA-BSA conjugate (×2, 100 ng) was immobilized on plate and incubated with 3C9C9 and varying concentration (0, 0.1, 1, 10, 100 and 1000 ng) of competing agents. 3C9C9 does not bind to normal nucleotides. FIG. 20B shows the reactivity of purified 3C9C9 mAb against selected DNA adducts by competitive ELISA. DHHεA-BSA conjugate (×2, 100 ng) was immobilized on plate and incubated with 3C9C9 and varying concentration (0, 0.1, 1, 10, 100 and 1000 ng) of competing agents. 3C9C9 does not bind to Acr-dG, 8-oxo-dG and cdA. Moderate binding to HNE-dG adducts was observed.

FIG. 21 shows the reactivity of purified 3C9C9 mAb against EH-modified calf thymus (CT) DNA and CT DNA by ELISA. DNA was immobilized on a plate (from 11 g to 0.1 ng DNA per well) and exposed to 3C9C9 mAb. One μg of EH-modified CT DNA contains 272.3 fmol of DHHεdA, whereas one g of unmodified CT DNA has 12.3 amol of adduct.

FIG. 22 shows detection of DHHεdA in EH-treated primary human hepatocyte nuclei by Fluorescence-activated cell sorting (FACS) analysis. There was a significant difference (t-test, p<0.05) between treatment (right hand column of each pair of columns) and control (left hand column of each pair of columns) groups at the time points 4, 8, 12 and 24 h. The significant decrease of DHHεdA in cells treated with EH at 24 h may be attributed to repair because less than about 20% cells underwent cell death.

### DESCRIPTION

γ-Hydroxy-1, N²-propanodeoxyguanosine (γ-OHPdG)

γ-Hydroxy-1, N²-propanodeoxyguanosine (γ-OHPdG) is a lipid peroxidation-derived mutagenic DNA adduct that is repaired by the nucleotide excision repair (NER) pathway. The structure of γ-OHPdG is set forth below as Formula I, wherein dR is 2-deoxy-D-ribose. γ-OHPdG is also known as Acr-dG3.

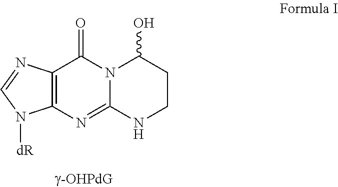

As set forth herein, a strong correlation was found between the levels of γ-hydroxy-1, N²-propanodeoxyguanosine (γ-OHPdG) in livers and the risk of hepatocellular carcinoma (HCC) in several animal models studied. Furthermore, using liver tumor specimens from HCC patients after surgery, it was also found that the higher levels of γ-OHPdG are strongly associated with low recurrence-free survival. These results demonstrate the potential of γ-OHPdG as a useful biomarker for HCC.

Provided herein is a method of reducing the recurrence of liver cancer in a subject comprising (a) obtaining a hepatic tissue sample from a subject (e.g., a subject with a history of treated liver cancer); (b) treating the subject for liver cancer; (c) determining a level of γ-hydroxy-1, N2-propanodeoxyguanosine (γ-OHPdG) in the sample; (d) comparing the detected level of step (c) to one or more control values, wherein an elevated level of γ-OHPdG indicates the subject is at risk for the recurrence of liver cancer; and (e) administering an antioxidant to the subject.

Also provided is a method of reducing the recurrence of liver cancer in a subject comprising (a) surgically treating a subject with liver cancer; (b) obtaining a hepatic tissue sample from a subject; (c) determining a level of γ-hydroxy-1, N²-propanodeoxyguanosine (γ-OHPdG) in the sample; (d) comparing the determined level of step (c) to one or more control values, wherein an elevated level of γ-OHPdG indicates the subject is at risk for the recurrence of liver cancer; and (e) administering an antioxidant to the subject.

Also provided is a method of diagnosing liver cancer in a subject comprising determining a level of γ-OHPdG in a sample from the subject, wherein an increased level of γ-OHPdG, as compared to a control, indicates that the subject has liver cancer or is at risk of developing liver cancer. A control can be, for example, the level of γ-OHPdG in a sample from a subject that does not have liver cancer, a level of γ-OHPdG in a sample from a subject that has been successfully treated for liver cancer, or a control value corresponding to a level of γ-OHPdG from a subject that does not have liver cancer or a subject that is not at increased risk, as compared to the general population, of developing liver cancer. Optionally, the control is from the same subject before cancer.

7-(1′,2′-dihydroxyheptyl)-1, N⁶-ethenodeoxyadenosine (DHHεdA)

Lipid peroxidation (LPO) of polyunsaturated fatty acids (PUFAs) is an endogenous source of α,β-unsaturated aldehydes that can produce a variety of cyclic DNA adducts. The mutagenic cyclic adducts derived from oxidation of 0-6 PUFAs may contribute to its cancer promoting activities. (E)-4-Hydroxy-2-nonenal (HNE) is a unique product of 0-6 PUFAs oxidation. HNE reacts with deoxyguanosine (dG) to form mutagenic 1,N²-propanodeoxyguanosine adducts (HNE-dG). It can also be oxidized to its epoxide (EH) and EH can react further with deoxyadenosine (dA) and dG forming 1 corresponding unsubstituted and substituted etheno adducts, such as 1,N⁶-ethenodeoxyadenosine (EdA), 1,N²-ethenodeoxyguanosine (1,N²-edG), 7-(1′,2′-dihydroxyheptyl)-1,N²-ethenodeoxyguanosine (DHHεdG) and 7-(1′,2′-dihydroxyheptyl)-1,N⁶-ethenodeoxyadenosine (DHHεdA). The structure of DHHεdA is set forth below as Formula II.

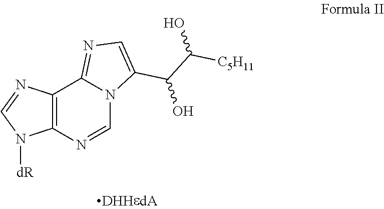

Provided herein is a method of reducing the recurrence of liver cancer in a subject comprising (a) obtaining a hepatic tissue sample from a subject; (b) treating the subject for liver cancer; (c) determining a level of 7-(1′,2′-dihydroxyheptyl)-1,N⁶-ethenodeoxyadenosine (DHHεdA) in the sample; (d) comparing the detected level of step (c) to one or more control values, wherein an elevated level of DHHεdA indicates the subject is at risk for the recurrence of liver cancer; and (e) administering an antioxidant to the subject.

Further provided is a method of reducing the recurrence of liver cancer in a subject comprising (a) surgically treating a subject with liver cancer; (b) obtaining a hepatic tissue sample from a subject; (c) determining a level of 7-(1′,2′-dihydroxyheptyl)-1,N⁶-ethenodeoxyadenosine (DHHεdA) in the sample; (d) comparing the determined level of step (c) to one or more control values, wherein an elevated level of DHHεdA indicates the subject is at risk for the recurrence of liver cancer; and (e) administering an antioxidant to the subject.

Provided herein is a method of diagnosing liver cancer in a subject comprising determining a level of 7-(1′,2′-dihydroxyheptyl)-1,N⁶-ethenodeoxyadenosine (DHHεdA) in a sample from the subject, wherein an increased level of DHHεdA, as compared to a control, indicates that the subject has liver cancer or is at risk of developing liver cancer.

Also provided is a method of diagnosing liver cancer in a subject comprising determining a level of 7-(1′,2′-dihydroxyheptyl)-1,N⁶-ethenodeoxyadenosine (DHHεdA) and/or a level of γ-hydroxy-1, N²-propanodeoxyguanosine (γ-OHPdG) in a sample from the subject, wherein an increased level of DHHεdA and/or an increased level of γ-OHPdG, as compared to a control, indicates that the subject has liver cancer or is at risk of developing liver cancer.

Further provided is a method of reducing the recurrence of liver cancer in a subject comprising (a) obtaining a hepatic tissue sample from a subject; treating the subject for liver cancer; (b) determining a level of 7-(1′,2′-dihydroxyheptyl)-1,N⁶-ethenodeoxyadenosine (DHHεdA) and/or a level of γ-hydroxy-1, N²-propanodeoxyguanosine (γ-OHPdG) in the sample; (c) comparing the detected level(s) of step (c) to one or more control values, wherein an elevated level of DHHεdA and/or an elevated level of γ-OHPdG indicates the subject is at risk for the recurrence of liver cancer; and (d) administering an antioxidant to the subject.

Also provided is a method of reducing the recurrence of liver cancer in a subject comprising (a) surgically treating a subject with liver cancer; (b) obtaining a hepatic tissue sample from a subject; (c) determining a level of 7-(1′,2′-dihydroxyheptyl)-1,N⁶-ethenodeoxyadenosine (DHHεdA) and/or a level of γ-hydroxy-1, N²-propanodeoxyguanosine (γ-OHPdG) in the sample; (d) comparing the determined level(s) of step (c) to one or more control values, wherein an elevated level of DHHεdA and/or an elevated level of γ-OHPdG indicates the subject is at risk for the recurrence of liver cancer; and (e) administering an antioxidant to the subject.

As used throughout, a control can be, for example, the level of DHHεdA or the level of γ-OHPdG in a sample from a subject that does not have liver cancer, a level of DHHεdA or the level of γ-OHPdG in a sample from a subject that has been successfully treated for liver cancer, or a control value corresponding to a level of DHHεdA or a level of γ-OHPdG from a subject that does not have liver cancer or a subject that is not at increased risk, as compared to the general population, of developing liver cancer. Optionally, the control is from the same subject before cancer.

Any of the methods provided herein can further comprise diagnosing the subject with liver cancer and treating the subject with liver cancer after diagnosis. Diagnosing a subject with liver cancer can include, one or more of a biopsy, CAT scan, angiogram, ultrasound, X ray, MRI, blood chemistry tests, assays of other molecular markers for liver cancer and the like. As used throughout, liver cancer can be hepatocellular carcinoma, fibrolamellar hepatocellular carcinoma, cholangiocarcinoma, angiosarcoma or secondary liver cancer that develops when primary cancer from another part of the body spreads to the liver. In some methods, the liver cancer is an early stage liver cancer, for example, a Stage I liver cancer where a single tumor has not grown into any blood vessels and the cancer has not spread to lymph nodes or distant sites. In other methods, the liver cancer is a Stage II, Stage III or Stage IV cancer. Any of the methods provided herein can further comprise a differential diagnosis that distinguishes liver cancer from other pathologies. For example, a diagnosis of fibrosis or cirrhosis of the liver can be confirmed or eliminated by a liver biopsy and/or by detecting serum levels of liver enzymes and albumin.

In the methods provided herein, the sample can be obtained from a subject with liver cancer or, alternatively, from a subject at risk of developing liver cancer. The sample can be, for example, a hepatic tissue sample, cells from a hepatic tissue sample, blood, plasma, serum, ascites fluid or urine from the subject. It is understood that the steps of obtaining a sample from the subject and treating the subject for liver cancer can be performed in any order. In other words, the sample can be obtained from the subject prior to or after treatment for liver cancer. The sample can also be obtained from the subject concurrently with treatment of the subject for liver cancer. Also, the step of determining the level of γ-OHPdG and/or the level of DHHεdA in the sample can be performed prior to or after treatment of liver cancer in the subject.

Risks associated with liver cancer include, but are not limited to, personal or family history of liver cancer, viral hepatitis (Hep-B and/or Hep-C), cirrhosis, non-alcoholic fatty liver disease, inherited metabolic diseases, obesity, alcohol use, obesity, type 2 diabetes, tyrosinemia, alpha1-antitrypsin deficiency, porphyria cutanea tarda, glycogen storage diseases, Wilson disease, exposure to aflatoxins, anabolic steroid use, parasitic infection and smoking.

As used herein, by reducing the recurrence of liver cancer is meant a method of preventing, precluding, delaying, averting, obviating, forestalling, stopping, or hindering the onset, incidence or severity of the reappearance of liver cancer in a subject. As utilized herein, by reappearance of liver cancer is meant the reappearance of one or more clinical symptoms of liver cancer after a period devoid of one or more clinical symptoms of liver cancer. The recurrence of liver cancer can be after treatment for liver cancer or after a remission. A recurrence can occur days, weeks, months or years after treatment or after a remission. For example, the disclosed method is considered to reduce the occurrence of liver cancer if there is a reduction or delay in onset, incidence or severity of the reappearance of liver cancer or one or more symptoms of liver cancer (e.g., abdominal pain, abdominal swelling, jaundice, enlarged liver, weight loss, hypercalcemia, hypoglycemia, gynecomastia erythrocytosis) in a subject at risk for a recurrence of liver cancer compared to control subjects at risk for a recurrence of liver cancer that did not receive an antioxidant. The disclosed method is also considered to reduce the recurrence of liver cancer if there is a reduction or delay in onset, incidence or severity of the reappearance of liver cancer, or one or more symptoms of liver cancer (e.g., abdominal pain, abdominal swelling, jaundice, enlarged liver, weight loss, hypercalcemia, hypoglycemia, gynecomastia erythrocytosis) in a subject at risk for recurrence of liver cancer after receiving an antioxidant as compared to the subject's progression prior to receiving treatment. Thus, the reduction or delay in onset, incidence or severity of recurrence of liver cancer can be about a 10, 20, 30, 40, 50, 60, 70, 80, 90, 100%, or any amount of reduction in between.

As used throughout, a subject can be a vertebrate, more specifically a mammal (e.g., a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, patient or subject may be used interchangeably and can refer to a subject with a disease or disorder (e.g., cancer). The term patient or subject includes human and veterinary subjects.

A utilized herein, a subject at risk for recurrence of liver cancer is a subject that is at risk for the reappearance of liver cancer after treatment for liver cancer or after remission from liver cancer. Treatment methods for liver cancer include, but are not limited to, surgery (for example, a partial hepatectomy, or a resection of liver cancer cells), ethanol injection therapy, implantation of alloactivated lymphocytes into or around the site of a liver tumor, a liver transplant, tumor ablation, tumor embolization, chemotherapy, radiation therapy, immunotherapy, targeted therapy with sorafenib or combinations of these treatment methods. In the methods provided herein, the level of γ-OHPdG and/or the level of DHHεdA in a sample from a subject treated for liver cancer can be determined or measured one day, two days, three days, four days, five days, six days, one week, two weeks, three weeks, one month, two months, three months, four months, five months six months or later, after treatment for liver cancer.

As used herein the terms treatment, treat, or treating refers to a method of reducing the effects of a disease or condition or symptom of the disease or condition. Thus in the disclosed method, treatment can refer to a 10%, 20%, 30%, 40%, 50%, 60%, 70%, 80%, 90%, or 100% reduction in the severity of an established disease or condition or symptom of the disease or condition. For example, treatment can refer to a 10%, 20%, 30%, 40%, 50%, 60%, 70%, 80%, 90%, or 100% reduction in tumor size. In another example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition, although treatment can include a cure or complete ablation of the disease, condition, or symptoms of the disease or condition.

Analytical techniques useful in determining the level of γ-OHPdG and/or the level of DHHεdA include, but are not limited to immunohistochemistry, immunoassay, radioimmunoassay (RIA), liquid chromatography mass spectrometry (LC-MS) techniques and surface plasmon resonance. In some methods an antibody that specifically detects γ-OHPdG and/or an antibody that detects DHHεdA is used. As used herein, the term antibody encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG and IgM. Several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called alpha, delta, epsilon, gamma, and mu, respectively. The term variable is used herein to describe certain portions of the antibody domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies. The constant domains are not involved directly in binding an antibody to an antigen but exhibit various effector functions, such as participation of the antibody in antibody-dependent cellular toxicity. Also included within the meaning of antibody or fragments thereof are conjugates of antibody fragments and antigen binding proteins (single chain antibodies) as described, for example, in U.S. Pat. No. 4,704,692, the contents of which are hereby incorporated by reference in their entirety.

Optionally, the antibody is a monoclonal antibody that specifically binds to γ-OHPdG or a monoclonal antibody that specifically binds to DHHεdA. The term monoclonal antibody as used herein refers to an antibody from a substantially homogeneous population of antibodies, i.e., the individual antibodies comprising the population are identical except for possible naturally occurring mutations that may be present in minor amounts. Monoclonal antibodies may be prepared using hybridoma methods, such as those described by Kohler and Milstein, Nature, 256:495 (1975), or Harlow and Lane, Antibodies: A Laboratory Manual, Cold Spring Harbor Publications, New York (1988). Monoclonal antibodies that specifically bind to and detect γ-OHPdG are available. See, for example, Pan et al., “Detection of acrolein-derived cyclic DNA adducts in human cells by monoclonal antibodies,” Chem. Res. Toxicol. 25(12): 2788-2795 (2012). As set forth in Example I, the levels of γ-OHPdG can also be determined using an immunohistochemistry techniques and a scoring system that allows one of skill in the art to determine whether a subject that has been or will be treated for liver cancer is at risk for a recurrence of liver cancer after treatment.

Provided herein is a monoclonal antibody that specifically binds to DHHεdA. By “specifically binding” or “selectively binding” is meant that the antibody binds to one agent (e.g., DHHεdA) or antigen to the partial or complete exclusion of other antigens or agents. By “binding” is meant a detectable binding at least about 1.5 times the background of the assay method. For selective or specific binding, such a detectable binding can be detected for a given antigen or agent but not a negative control antigen or agent.

In some examples, a monoclonal antibody that specifically binds to DHHεdA does not bind to nucleotides (for example, dA, dC, dG and/or T), or other cyclic adducts (for example, 8-oxo-dG, γ-OHPdG, α-OHPdG (Acr-dG1/2), HNE-dG and/or edA), to any significant extent. In some examples, the monoclonal antibody has at least ten times, twenty-five times, fifty times or one hundred times stronger affinity towards DHHεdA than another DNA cyclic adduct, for example, HNE-dG, 8-oxo-dG, γ-OHPdG, α-OHPdG, HNE-dG or edA. Examples of monoclonal antibodies that specifically bind to DHHεdA and have at least one hundred times stronger affinity towards DHHεdA than HNE-dG include, but are not limited to, the monoclonal antibodies produced by hybridoma cell line 3C3B6, 3C3E12, 3C9C9, 3C9G2, 4E10B8, and 4E10F2.

Also provided are hybridoma cell lines 3C3B6, 3C3E12, 3C9C9, 3C9G2, 4E10B8, and 4E10F2. Further provided is monoclonal antibody 3C3B6, produced by hybridoma cell line 3C3B6, monoclonal antibody 3C3E12, produced by hybridoma cell line 3C3E12, monoclonal antibody 3C9C9, produced by hybridoma cell line 3C9C9, monoclonal antibody 3C9G2, produced by hybridoma cell line 3C9G2, monoclonal antibody 4E10B8, produced by hybridoma cell line 4E10B8, and monoclonal antibody 4E10F2, produced by hybridoma cell line 4E10F2. Also provided is a monoclonal antibody having the same epitope specificity as an antibody produced by a disclosed hybridoma cell line. Humanized or human versions of antibodies 3C3B6, 3C3E12, 3C9C9, 3C9G2, 4E10B8, and 4E10F2 are also provided. Optionally, the humanized or human antibody comprises at least one complementarity determining region (CDR) or all CDRs of an antibody having the same epitope/hapten specificity as an antibody produced by a disclosed hybridoma cell line. As used herein, the term epitope/hapten is meant to include any determinant capable of specific interaction with the provided antibodies. Epitopic or Haptenic determinants usually consist of chemically active surface groupings of molecules and usually have specific three dimensional structural characteristics, as well as specific charge characteristics.

According to the methods taught herein, the subject is administered an effective amount of an antioxidant, such as, for example, one or more of Theaphenon E, Polyphenone E, vitamin E, α-lipoic acid, or derivatives thereof. The terms effective amount and effective dosage are used interchangeably. The term effective amount is defined as any amount necessary to produce a desired physiologic response. Effective amounts and schedules for administering the agent may be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, toxicity and the like. Generally, the dosage will vary with the activity of the specific compound employed, the metabolic stability and length of action of that compound, the species, age, body weight, general health, sex and diet of the subject, the mode and time of administration, rate of excretion, drug combination, and severity of the particular condition and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

Any appropriate route of administration may be employed, for example, parenteral, intravenous, subcutaneous, intramuscular, intraventricular, intracorporeal, intraperitoneal, rectal, or oral administration. Administration can be systemic or local. Pharmaceutical compositions can be delivered locally to the area in need of treatment. Multiple administrations and/or dosages can also be used. Effective doses can be extrapolated from dose-response curves derived from in vitro or animal model test systems.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### Example I

γ-Hydroxy-1, N²-propanodeoxyguanosine (γ-OHPdG) is a lipid peroxidation-derived mutagenic DNA adduct that is repaired by the nucleotide excision repair (NER) pathway. Xeroderma pigmentosum group A (Xpa) knockout mice deficient in NER developed a high incidence of spontaneous liver tumors with more than 85% of GC>TA somatic mutations. It was hypothesized that accumulation of the endogenous γ-OHPdG plays a role in liver tumorigenesis of Xpa^{-/-} mice, and its suppression by antioxidants can result in significant liver cancer prevention. Levels of γ-OHPdG showed an age-dependent increase, and they were higher in the livers of Xpa^{-/-} mice than the WT mice. Theaphenon E and α-lipoic acid, but not vitamin E, effectively decreased γ-OHPdG levels in the liver DNA of Xpa^{-/-} mice and both compounds reduced HCC incidence to 14% and 65%, respectively, from 100% in the untreated controls. Using other models with high risk of HCC, γ-OHPdG was also found to be elevated in the livers of the DEN-treated mice and the Long-Evans Cinnamon (LEC) rats. Again, Theaphenon E effectively inhibited HCC formation in the DEN-induced HCC model. Whether γ-OHPdG serves as a biomarker of HCC recurrence using HCC specimens from 45 patients. The study revealed that higher levels of γ-OHPdG are strongly associated (p<0.007) with low recurrence-free survival. Together, these results suggest γ-OHPdG is a biologically relevant biomarker for predicting the risk of HCC.

### Chemicals and Enzymes

All reagents purchased were analytical or HPLC grade. HPLC grade acetonitrile (ACN) and methanol (MeOH) were purchased from EMD (EMD Chemicals, Gibbstown, N.J.). Alkaline phosphatase grade I from calf intestine was purchased from Roche (Roche Applied Science, Indianapolis, Ind.). Acrolein was purchased from Alfa Aesar (Alfa Aesar, Ward Hill, Mass.). [¹³C₁₀,¹⁵N₅]-2′-Deoxyguanosine was obtained from Spectra Stable Isotopes (Cambridge Isotope Laboratories, Andover, Mass.). 2′-Deoxyguanosine monohydrate, ion chromatography grade heptafluorobutyric acid (HFBA), 10M ammonium formate solution, deoxyribonuclease I from bovine pancreas Type II (DNase I), and purified phosphodiesterase I from Crotalus adamanteus venom were purchased from Sigma (Sigma-Aldrich Corp., St. Louis, Mo.). Adenosine deaminase from calf spleen was from Worthington (Worthington Biochemical Corp., Lakewood, N.J.), DEN (Sigma-Aldrich, St. Louis, Mo.). All other reagents used were analytical or HPLC grade. Vitamin E and α-lipoic acid were purchased from ThermoFisher (Walthan, Mass.), Theaphenon E was kindly provided by Dr. Yukihiko Hara.

### Animals

C3H/HeN.Xpa^{-/-} mice were created by crossing C57/B6. Xpa^{-/-} mice with C3H/HeN mice. The resulting heterozygous mice were then backcrossed to C3H mice for five generations. Mice homogeneous for the Xpa-null allele on a C3H background were subsequently generated by brother-sister mating. All of the mice were housed in a temperature-controlled, light-regulated space with 12-hour light and dark cycles and were given unrestricted access to food and water throughout the experiments. The protocol used in this study was approved by Georgetown University Animal Care and Use Committee. The animals were fed with AIN-76A powder diets obtained from Dyets, Inc. (Bethlehem, Pa.) at the age of 4 weeks; four types of diets (control, α-lipoic acid, Theaphenon E and vitamin-E) were used in this study. The only difference between the diets is the sucrose content to be replaced by the antioxidant (Table 1: the doses of the antioxidants, α-lipoic acid (2 g/kg), Theaphenon E (20 g/kg) and vitamin E (1.8 g/kg)), were selected according to recent studies, respectively. The nutrition for all the four diets was calculated within the range of 3694 to 3766 kcal/kg. (n=6-7 for time dependent DNA adduct assay, n=18-19 for tumor bioassay after 72 weeks under different diets (see Table 4)). C57/B6 mice at the age of 10 days were obtained from Charles River Laboratories, Inc. (Wilmington, Mass.). Two-week old C57/B6 mice were injected intraperitoneally with 5 mg/kg DEN. LEC rats at the age of 4 weeks were obtained from Charles River Japan (Yokohama, Japan.). LEC rats (n=12) were sacrificed after 12 and 20 weeks. The liver tissues were dissected and kept at −80° C. until usage for DNA isolation.

TABLE 1

| | Control | DHLA | Theaphenon E | Vitamin E |
|---|---|---|---|---|
| Ingredient | (g/kg) | (g/kg) | (g/kg) | (g/kg) |
| DHLA (g/kg | 0 | 2 | 0 | 0 |
| Theaphenon-E | 0 | 0 | 20 | 0 |
| Vitamin-E | 0 | 0 | 0 | 1.8 |
| Casein | 200 | 200 | 200 | 200 |
| Sucrose | 500 | 498 | 480 | 498.2 |
| Cornstarch | 150 | 150 | 150 | 150 |
| DL-Methionine | 3 | 3 | 3 | 3 |
| Cellulose | 50 | 50 | 50 | 50 |
| Corn Oil | 50 | 50 | 50 | 50 |
| Mineral Mix | 35 | 35 | 35 | 35 |
| Vitamine Mix | 10 | 10 | 10 | 10 |
| Choline | 2 | 2 | 2 | 2 |
| Bitartrate | | | | |

[symbol]

uantification of γ-OHPdG in DNA by LC-MS/MS

DNA (100-500 μg) was dissolved in 5 mM magnesium chloride (800 μL per mg DNA) and spiked with 100 fmol of [¹³C₁₀,¹⁵N₅]-γ-OHPdG and 50 fmol of [¹³C₁₀,¹⁵N₅]−/− OHPdG. Enzymatic hydrolysis was performed by incubation with DNase I (1300 units/mg DNA) at 37° C. for 30 min followed by a second addition of DNase I (1300 units/mg DNA). After an additional 10 min at 37° C., alkaline phosphatase (380 units/mg DNA), phosphodiesterase I (0.06 units/mg DNA) and adenosine deaminase (0.5 units) were added, and the mixture was incubated for 1 h. After digestion a small aliquot of hydrolysate was used for dG quantification and the remaining digest was purified by solid phase extraction (SPE) using polymeric C18, 30 mg/1 mL, Strata-X columns (Phenomenex, Inc., Torrance, Calif.). Prior to use, the columns were conditioned by ACN (3×1 mL) and 25 mM ammonium formate buffer pH=4 (3×1 mL). After sample loading, the columns were washed with 2.5% ACN in 25 mM ammonium formate buffer pH=4 (1×1 mL). Then, γ-OHPdG adducts were collected by eluting with 5% ACN in 25 mM ammonium formate buffer pH=4 (1×1 mL). Samples were dried over vacuum using a SpeedVac and kept at −80° C. Before quantification assay samples were reconstituted with 50 μl of water, and 37 μL of sample was injected onto LC-MS/MS.

### γ-OHPdG Quantification by LC-MS/MS

Detection and quantification of γ-OHPdG adducts was carried on AB SCIEX 6500 QTrap triple quadrupole mass spectrometer (SCIEX, Framingham, Mass.) coupled with an ACQUITY UPLC liquid chromatography system (Waters Corporation, Milford, Mass.) equipped with 50×2.1 mm, 1.7 m particle size C18 column (Waters ACQUITY UPLC BEH C18). The separation of adducts was performed isocratically by eluting with 3% ACN, 1 mM ammonium formate buffer over 3.5 min using 0.5 mL/min flow rate at 40° C., followed by 100% ACN wash. The ESI source operated in positive mode. The multiple reaction monitoring (MRM) experiment was performed using ion transitions of 324.2-208.1 m/z (γ-OHPdG) and 339.2-218.1 m/z ([¹³C₁₀,¹⁵N₅]-γ-OHPdG) with a collision energy (CE) of 20 eV for quantification, and those of 324.2-190.1 m/z (γ-OHPdG) and 339.2-200.1 m/z ([¹³C₁₀,¹⁵N₅]-γ-OHPdG) with a CE of 47 eV for structural confirmation. All other parameters were optimized to achieve maximum signal intensity. Calibration curves were constructed for all the isomers before each analysis using standard solutions of γ-OHPdG and [¹³C₁₀,¹⁵N₅]-γ-OHPdG. A constant concentration of [¹³C₁₀,¹⁵N₅]-γ-OHPdG (1 fmol/μl) was used with different concentrations of γ-OHPdG (1.68 amol/μl-220 fmol/μl) and analyzed by LC-MS/MS-MRM using 37 μl injections.

### Patients and Database

Paraffin-embedded liver biopsy or HCC specimens from patients who had liver biopsies or curative resection of HCC as part of standard medical care were obtained from Georgetown University Medical Center. Informed consent was obtained from all patients under an approved IRB protocol (#1992-048). A secure database with proper safeguards was constructed for the management of patient data. An Institutional Review Board approved the ethical, legal, and social implications of the project.

### Whole Exome Sequencing

Genomic DNA extraction, library preparation, and whole exome sequencing was performed by Otogenetics (Norcross, Ga., USA). The genomic mouse DNA samples were constructed into libraries and hybridized to Agilent SureSelect Mouse All Exon 51 Mb probes (Santa Clara, Calif., USA). Paired end sequencing was executed on the Illumina HiSeq 2500 (San Diego, Calif., USA) and raw data files were pushed through the DNAnexus standard exome analysis pipeline to complete alignment, quality control, coverage analysis, and variant calling.

### Immunohistochemical Staining

Immunohistochemical detection of γ-OHPdG in healthy and cancerous tissues was performed by using tissue microarrays (HLiv-HCC060CD-01 and HLiv-HCC180Sur-02, US Biomax, Rockville, Md.), PFA-fixed and paraffin-embedded blocks of tumor tissue from subjects operated upon at Georgetown University. All patients gave informed consent, and the study was authorized by the respective Hospital Ethics Committees. Immunoscores were calculated by adding intensity and region of staining: 0, 1, 2, 3 correspond to no, weak, moderate, and high intensity staining; 0, 1, 2, 3 correspond to no, focal, regional, and diffuse region staining. More specifically, immunohistochemical staining of liver was performed for Acrolein-dG. Five micron sections from formalin fixed paraffin embedded tissues were de-paraffinized with xylenes and rehydrated through a graded alcohol series. Heat induced epitope retrieval (HIER) was performed by immersing the tissue sections at 98° C. for 20 minutes in 110 mM Tris, 1 m M EDTA pH 9.0 buffer (Genemed). Immunohistochemical staining was performed using a horseradish peroxidase labeled polymer from Dako (K4001) according to manufacturer's instructions. Briefly, slides were treated with 3% hydrogen peroxide and 10% normal goat serum for 10 minutes each, and exposed to primary antibodies for Acr-dG (1:500) for 1 hr at room temperature. Slides were exposed to the mouse HRP labeled polymer for 30 min and DAB chromagen (Dako) for 5 minutes. Slides were counterstained with Hematoxylin (Fisher, Harris Modified Hematoxylin), blued in 1% ammonium hydroxide, dehydrated, and mounted with Acrymount. Consecutive sections with the primary antibody omitted were used as negative controls.

### Data Analysis

The variant files (.vcf) provided by Otogenetics (Norcross, Ga., USA) were processed through Ensembl's Variant Effect Predictor (VEP) tool. VEP is an online tool that annotates variants and provides a statistical summary. The files were annotated with the mouse reference genome 10 (GRCm38.p4 assembly), and the RefSeq transcript and dbSNP database. In each tumor-normal pair, variants found in both the normal and tumor tissue were removed to reveal variants found only in the tumor tissue. Each variant location was then manually viewed concurrently in the Integrative Genomics Viewer (IGV, Broad Institute, Cambridge, Mass., USA) between the paired normal and tumor tissue for visual inspection and quality control.

### Statistical Analysis

The differences in adduct levels were compared using Student's t test. Fisher's exact test was used in analyzing categorical data. Kaplan-Meier method was used to analyze the survival data and Log-rank test was used to compare the survival between different groups. Z-test was employed to calculate the p-values of tumor incidences. P-values <0.05 were considered statistically significant. The SAS software (SAS Inc, Cary, N.C.) version 9.3 was used for statistical analysis.

Increased Levels of Mutagenic γ-OHPdG is Associated with Hepatocarcinogenesis in Xpa^{-/-} Mice

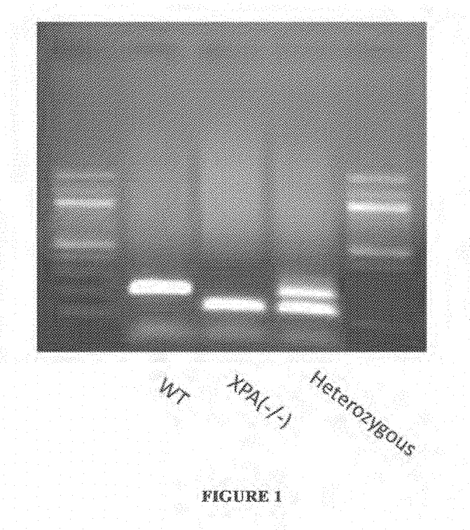

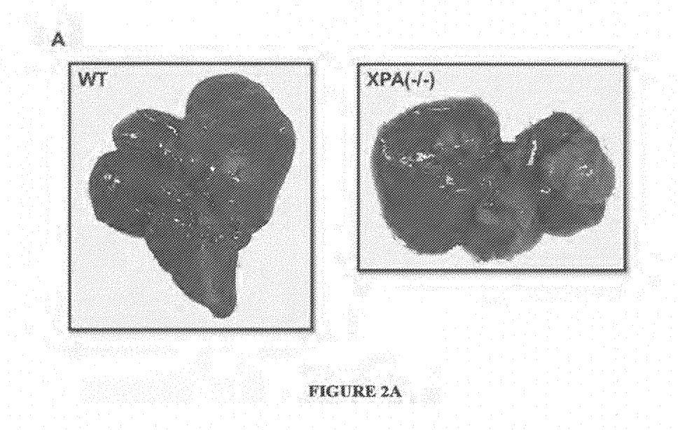

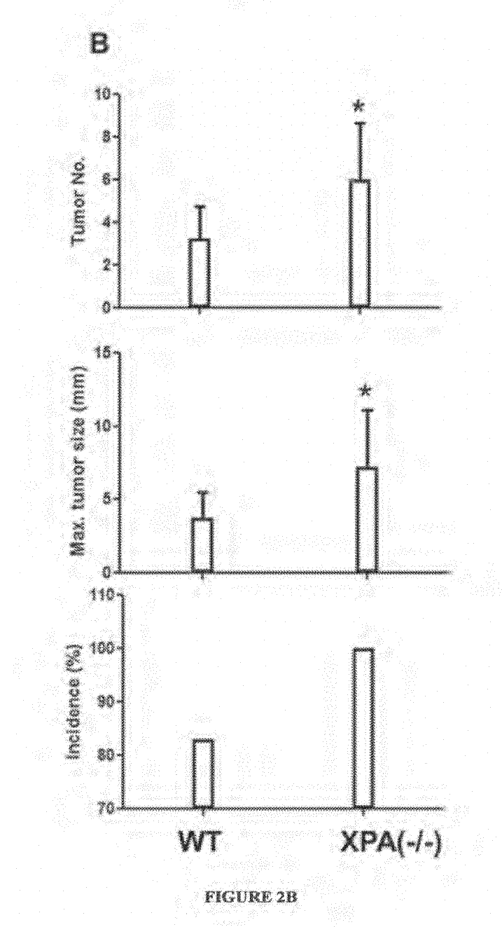

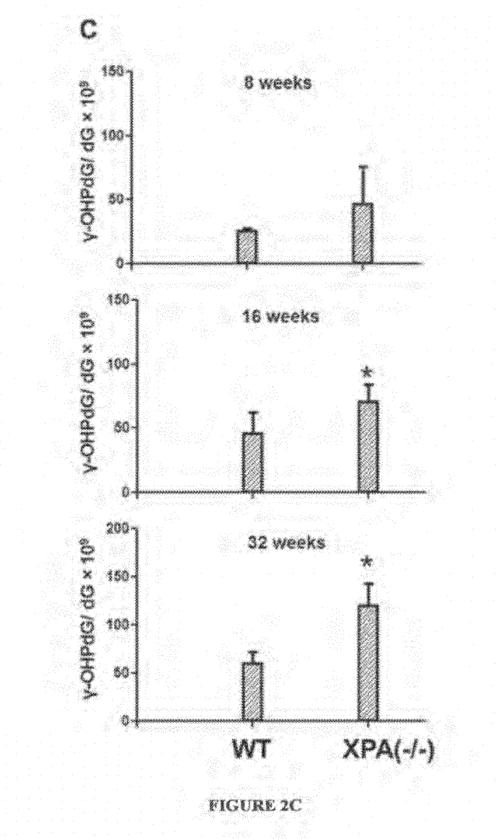

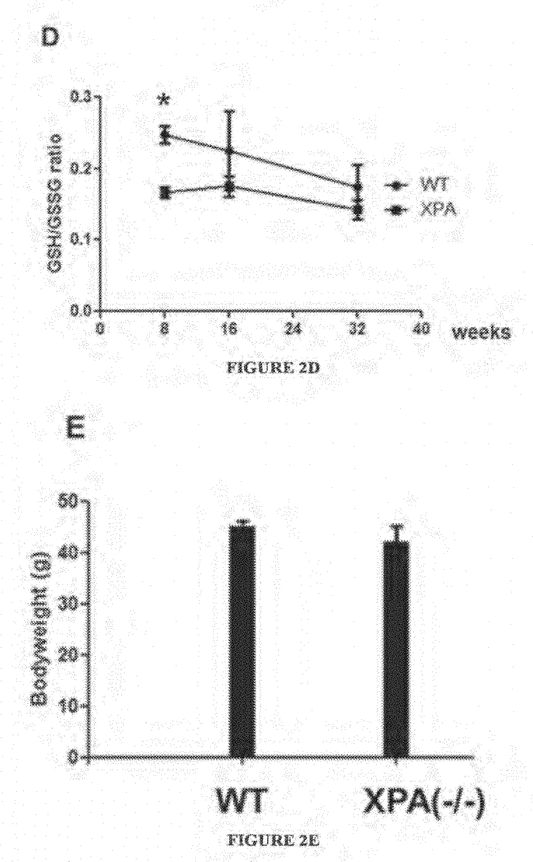

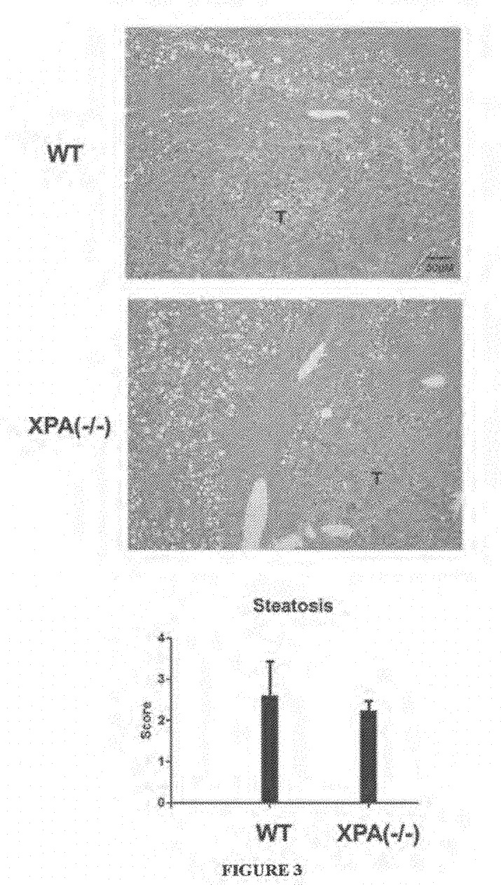

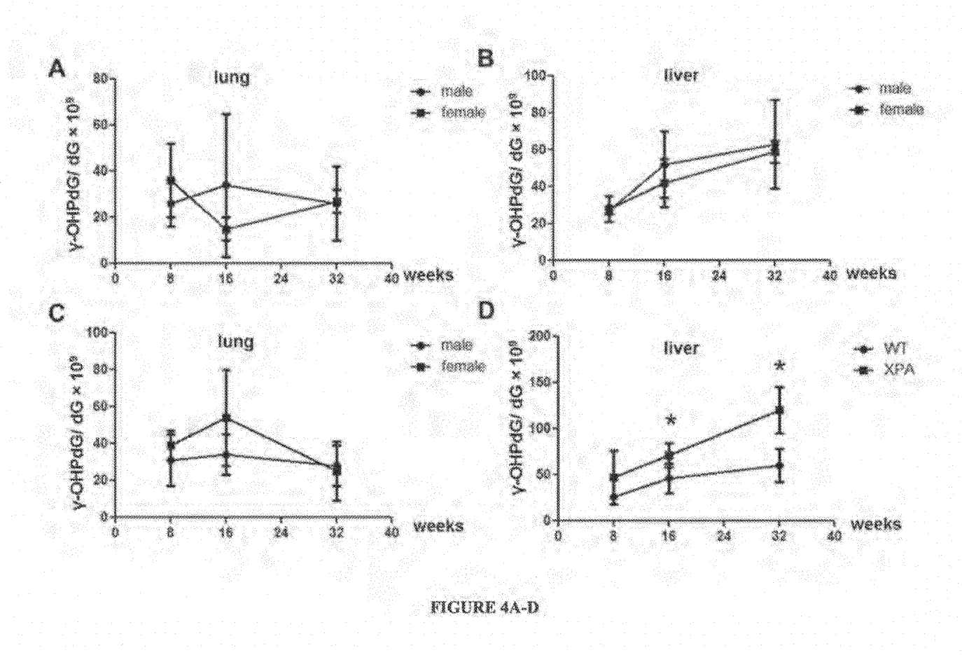

The mutagenicity of γ-OHPdG has been reported in in vitro models, but the role of γ-OHPdG with in vivo carcinogenesis was lacking. To employ genetic tools of next-generation sequencing (NGS), this work was mainly conducted in mice. First, the association between γ-OHPdG and liver tumorigenesis in Xpa^{-/-} mice deficient in NER was examined. γ-OHPdG is the only known endogenously formed DNA adduct repaired by NER, which is responsible for the removal of bulky, DNA helix-distorting adducts. Xpa^{-/-} C57/B6 mice are primarily used as a skin-cancer model when exposed to ultraviolet (UV)-B irradiation, however, they also develop spontaneous liver tumors. It was proposed that the cumulated γ-OHPdG, due to the lack of repair, contributes to the hepatocarcinogenesis in this model. Xpa^{-/-} C57/B6 mice were back-crossed with C3H/HeNCrl mice, which have a high rate of spontaneous liver cancer (FIG. 1). Compared to WT controls, Xpa^{-/-} mice not only had a higher incidence of liver tumors but also showed significantly larger tumor sizes and increased multiplicity (FIGS. 2A and 2B). The liver nodules from WT and Xpa^{-/-} mice were determined to be malignant based on tissue architecture and cytologic atypia, including abnormal trabeculi, nuclear pleomorphism, and absence of normal histology (FIG. 3). The γ-OHPdG levels increased age-dependently in Xpa^{-/-} mice from age 8-week to 32-week (p<0.05, FIG. 2C). Mouse gender had no influence on the γ-OHPdG levels and the tumor size and multiplicity (FIG. 4). A statistically significant difference in the hepatic levels of γ-OHPdG and liver cancer development between Xpa^{-/-} and WT mice was detected (FIG. 2).

LEC rats are inflicted with increased LPO due to abnormal copper accumulation, mimicking that of human Wilson's disease. As a result, LEC rats develop acute hepatitis, followed by chronic hepatitis, and eventually HCC. As shown in FIG. 5, γ-OHPdG levels in the livers of LEC rats were significantly higher than that of the WT Long Evans (LE) rats. These results provide additional support of the elevated γ-OHPdG levels in the livers of animals with increased risk of HCC.

### GC>TA is the Dominant Somatic Mutation in HCC of Xpa^{-/-} Mice

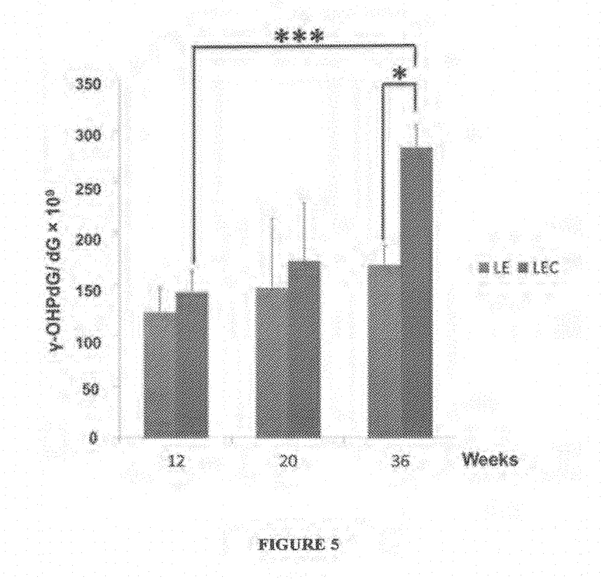

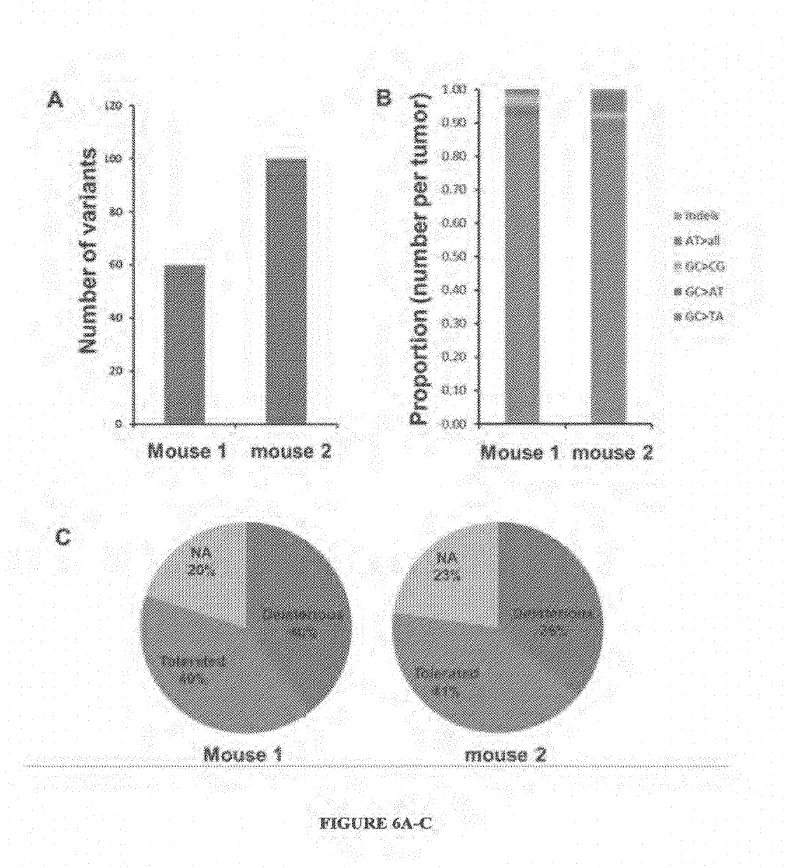

γ-OHPdG causes GC>TA and GC>AT mutations. It was hypothesized that the increased γ-OHPdG in the livers of Xpa^{-/-} mice can lead to a somatic mutation pattern in which GC>TA and GC>AT mutations are the most frequent alterations. Mutation frequencies in two pairs of liver tumor nodules versus adjacent normal liver tissues from two Xpa^{-/-} mice were compared using whole exome next-generation sequencing (NGS). Whole exome sequencing produced a mean yield of 23.8 million reads or 2.5 gigabases (Gb) of data per sample with 94.1%>Q30. The samples were sequenced to a mean coverage of 29×, and 99.4% of the reads were mapped to the target regions. 60 and 100 variants were found in the two liver nodules (FIG. 6A), with GC>TA mutation as the dominant alteration accounting for 92% and 86% mutations, respectively (FIG. 6B). While examining the Sorting Intolerant from Tolerant (SIFT) prediction scores, it was also noted that more than 35% of the variants in both samples were predicted as deleterious mutations (FIG. 6C). The high GC>TA mutation frequency in the Xpa^{-/-} mouse liver cancers suggests that γ-OHPdG plays a role in the mutagenesis of HCC development. Different from other solid tumors in which CG>TA transitions are the highest variations in the mutation spectrum, variants in human HCC also showed an over-representation of GC>TA transversion. A number of mutant genes were identified within mouse liver nodules that were reported in human HCC, including ABCA1, CSMD1, LAMA2, TRRAP, and TRANKI, suggesting this model is relevant to human liver carcinogenesis.

### Antioxidants Suppress Liver γ-OHPdG Levels and HCC Development in Xpa^{-/-} Mice

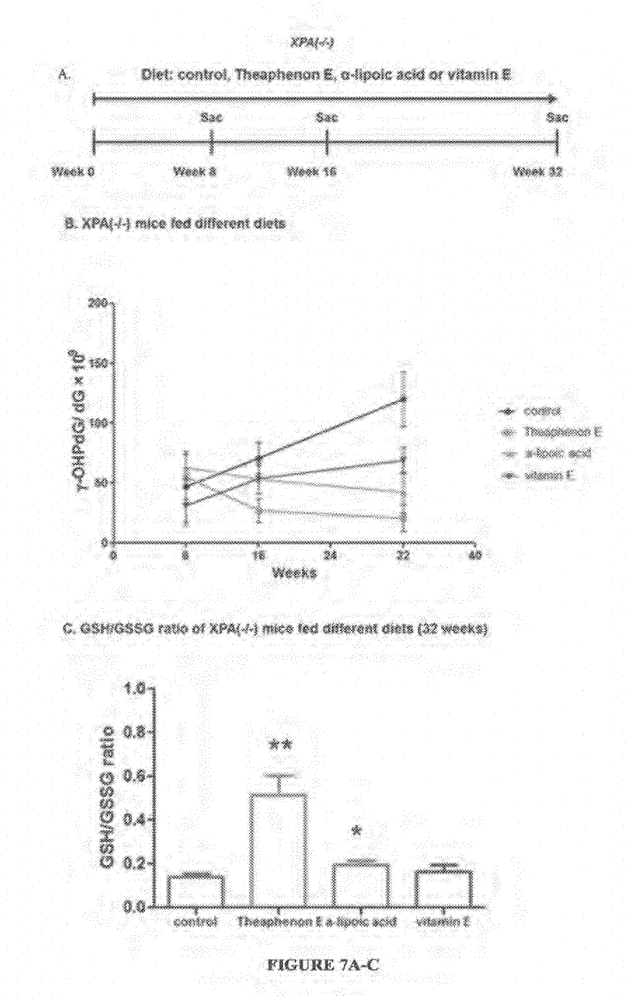

To further assess the role of γ-OHPdG in HCC development, whether blockage of γ-OHPdG formation in the livers of Xpa^{-/-} mice would reduce HCC incidence was determined. Xpa^{-/-} mice yield 100% liver cancer incidence at the end of the 72-week bioassay. Three antioxidants (Theaphenon E, α-lipoic acid, and vitamin E) known to suppress LPO were used. To determine whether these antioxidants suppress the liver γ-OHPdG levels, Xpa^{-/-} mice were fed diets containing antioxidants (details of the bioassay and diet components are set forth above). A significant decrease of liver γ-OHPdG levels was observed in the mice fed the antioxidant diets, with different potencies: Theaphenon E>α-lipoic acid >vitamin E (FIG. 7), whereas no significant changes of the γ-OHPdG levels were found in the lungs, a non-target organ. The ratio of reduced to oxidized glutathione (GSH/GSSG), an indicator of oxidative stress was also examined. The increases in the ratio of GSH/GSSG in the liver tissues from the mice fed different antioxidants for 32 weeks are consistent with the decreases of γ-OHPdG (FIG. 7C).

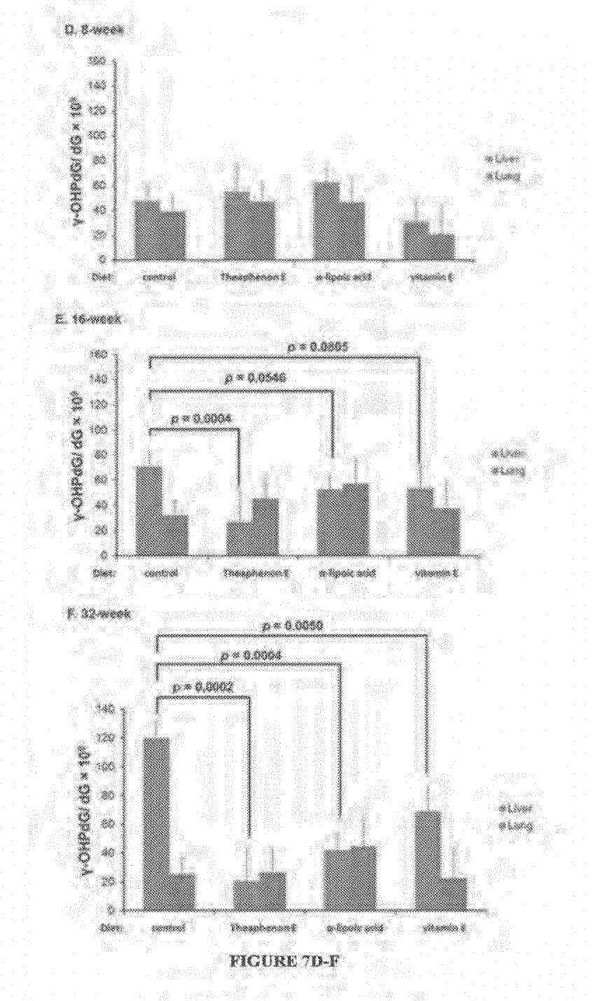

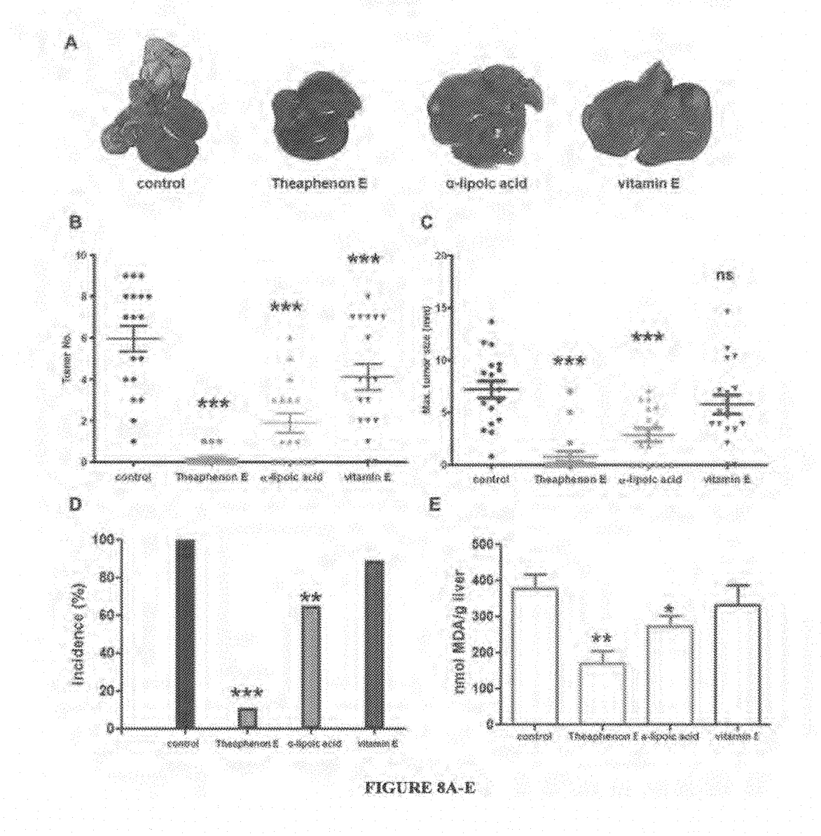

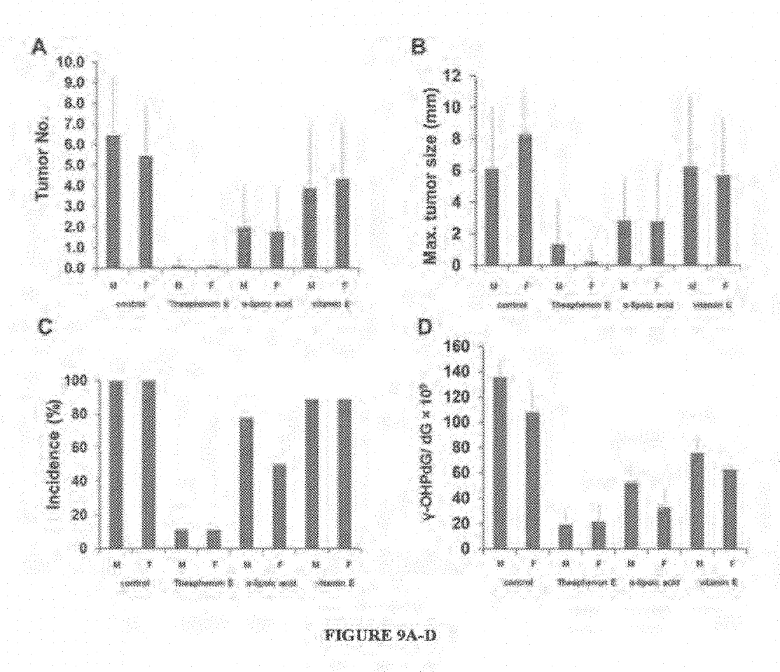

Having demonstrated that antioxidants can suppress γ-OHPdG formation, the relationships between γ-OHPdG and hepatocarcinogenesis were examined. Xpa^{-/-} mice were fed with four types of diets (control, Theaphenon E, α-lipoic acid, and vitamin E) for 72 weeks. Theaphenon E showed the strongest inhibition of hepatocarcinogenesis by reducing HCC incidence from 100% in mice on control diet to 14%. α-Lipoic acid resulted in a decrease of HCC incidence to 65%, whereas vitamin E had no significant effect (FIG. 8). The potency of tumor inhibition by the antioxidants showed a strong correlation with the liver γ-OHPdG levels in these mice. The protective effects of antioxidants against HCC were similar in both male and female mice (FIG. 9), in agreement with the effects on γ-OHPdG suppression in both genders (FIG. 4). The potency of inhibiting LPO by the antioxidants, determined by measuring the MDA level in mouse livers, is consistent with that of γ-OHPdG (FIG. 8E). Although a significant loss in body weight gains was noted in the Theaphenon E group (FIG. 10), no food consumption difference was found and the mice in this group were leaner and appeared healthy (FIG. 10C). This is probably related to thermogenesis, fat oxidation, and sparing fat free mass effects known to be caused by green tea extract.

γ-OHPdG Levels Correlate with Hepatocarcinogenesis in the DEN-Induced HCC Mouse Model

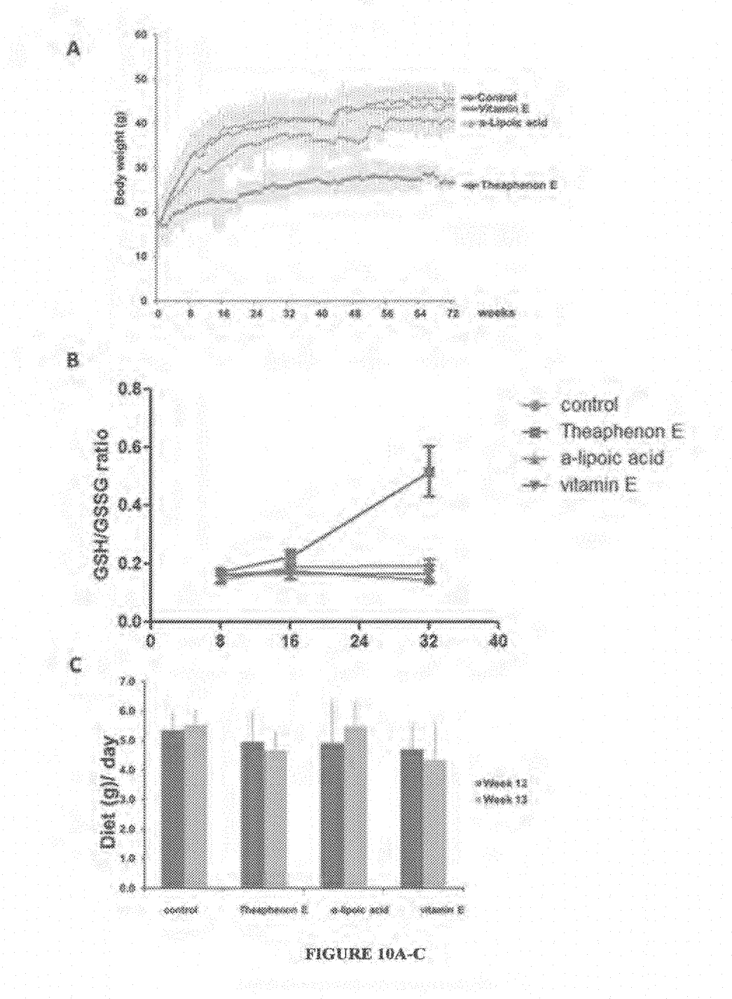

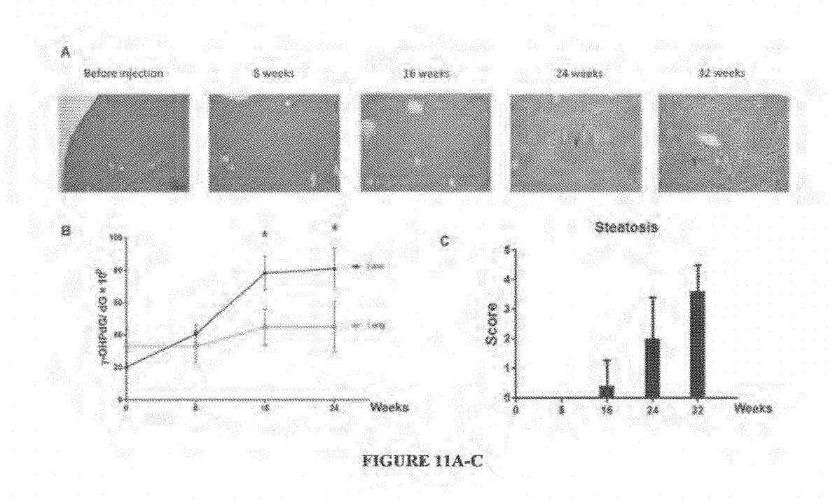

The relationship of γ-OHPdG in HCC was further examined in C57/B6 mice involving a single injection of procarcinogen DEN. In this model, mice develop poorly differentiated HCC nodules within 32 to 36 weeks after DEN exposure. DEN (5 mg/kg) was administered to male mice on postnatal day 14, and placed the mice on AIN-76A powder diet with and without Theaphenon E one week later. A time-dependent increase of steatosis was observed in the mice under control diet. Liver nodules were observed after eight months (FIG. 11). They were determined to be malignant based on architectural and cytologic atypia, including abnormal trabeculae, nuclear pleomorphism, and absence of normal histologic landmarks. To test whether γ-OHPdG levels are associated with hepatocarcinogenesis, γ-OHPdG was measured in the livers obtained at four time intervals, shortly before and, 8, 16, and 24 weeks after DEN-injection. An age-dependent increase of γ-OHPdG was seen in the livers, but not in the lungs (a non-target tissue) (FIG. 11B). These results are consistent with previous reports that DEN can cause oxidative stress and induces LPO. Whether antioxidant can suppress HCC development in this model was tested. Similar to the results in Xpa^{-/-} mice, Theaphenon E showed a remarkable suppression of HCC formation in these mice, decreasing tumor incidence from 100% to 40%, tumor size from 10 mm to <1 mm, and multiplicity from 30 to 3 nodules per mouse (FIG. 12).

### γ-OHPdG as a Prognostic Biomarker for HCC Recurrence in Patients

The data described above demonstrated that γ-OHPdG is closely associated with liver carcinogenesis in the animal models. To investigate the role of γ-OHPdG in human HCC, forty human liver samples were procured with different pathology diagnoses, including two normal livers, seven livers with cirrhosis, three with cirrhosis and hyperplasia, four with hyperplasia, and twenty-two HCC tissues. They were stained with a γ-OHPdG monoclonal antibody (Pan et al., “Detection of acrolein-derived cyclic DNA adducts in human cells by monoclonal antibodies,” Chem. Res. Toxicol. 25(12): 2788-2795 (2012)). The association between pathology and immunoscore of γ-OHPdG was examined using Fisher test. A significant association was found between liver disease progression and the immunoscore of γ-OHPdG with p=0.0364 (Table 2).

TABLE 2
Association between pathology and immunoscore of γ-OHPdG

| | Table of Sum_Score by pathology | | | | | | |
|---|---|---|---|---|---|---|---|
| | | pathology | | | | | |
| | | | | | Liver cirrhosis | | |
| Frequency | Sum_Score | HCC | Hyperplasia | Liver cirrhosis | with hyperplasia | Normal tissue | Total |
| | 0 | 1 | 0 | 0 | 1 | 0 | 2 |
| | 2 | 1 | 0 | 4 | 0 | 0 | 5 |
| | 3 | 4 | 2 | 2 | 0 | 0 | 8 |
| | 4 | 3 | 1 | 0 | 1 | 2 | 7 |
| | 5 | 8 | 1 | 1 | 1 | 0 | 11 |
| | 6 | 5 | 0 | 0 | 0 | 0 | 5 |
| | Total | 22 | 4 | 7 | 3 | 2 | 38 |

TABLE 3
Association between stage of HCC and immunoscore of γ-OHPdG

| | Table of stage by Sum_Score | | | | | | | |
|---|---|---|---|---|---|---|---|---|
| | | Sum_Score | | | | | | |
| Frequency | stage | 0 | 2 | 3 | 4 | 5 | 6 | Total |
| | I | 0 | 0 | 1 | 0 | 0 | 2 | 3 |
| | II | 0 | 0 | 0 | 1 | 3 | 0 | 4 |
| | III | 1 | 1 | 1 | 0 | 2 | 0 | 5 |
| | IV | 0 | 0 | 2 | 2 | 3 | 3 | 10 |
| | Total | 1 | 1 | 4 | 3 | 8 | 5 | 22 |

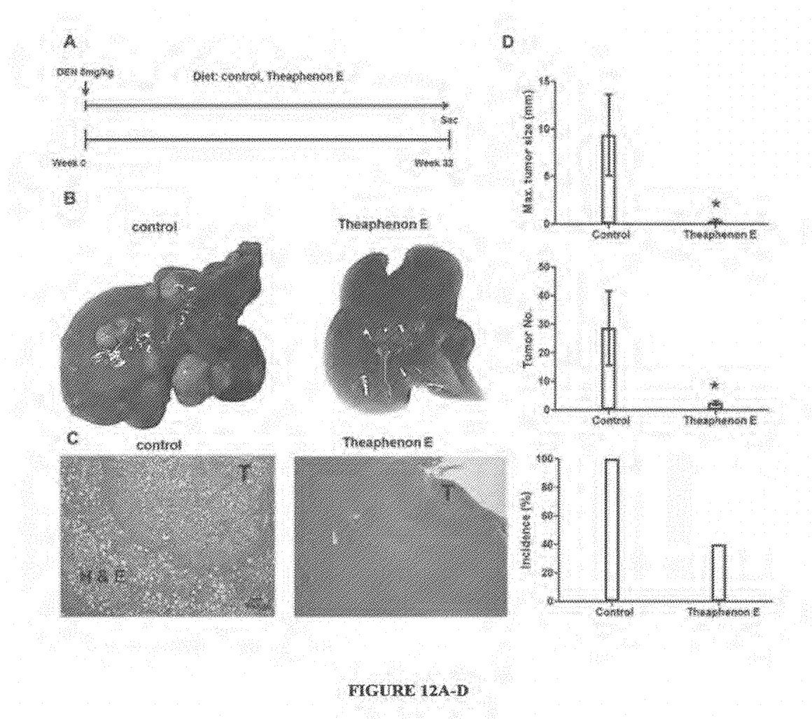

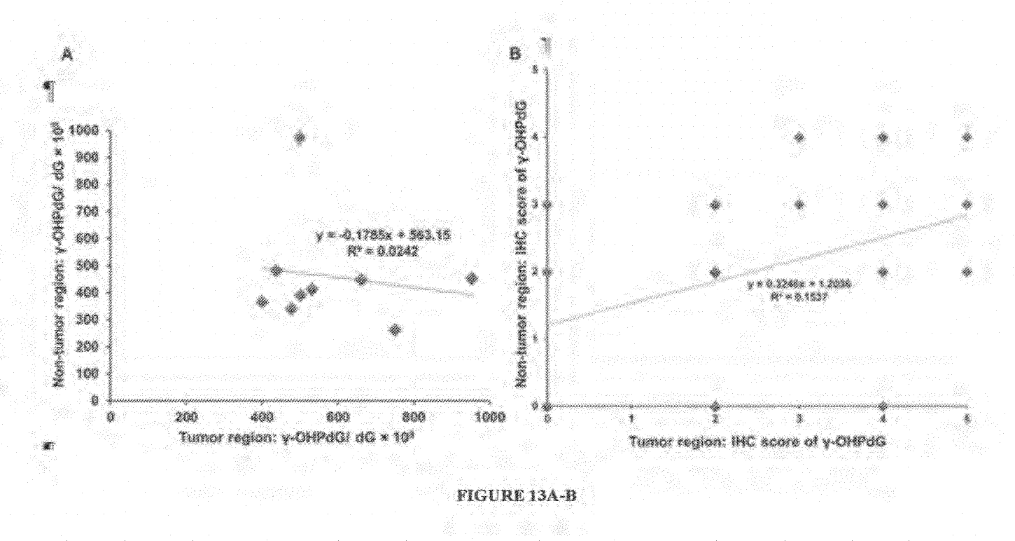

Next, whether there is a correlation of γ-OHPdG levels between tumor and adjacent tissue was examined. Liver samples from mice, including nine pairs of tumor and adjacent tissues (3 of WT, 3 of Xpa^{-/-} fed control diet, and 3 of Xpa^{-/-} fed α-lipoic acid diet), were used. No correlation was found between tumor and adjacent normal tissue in the levels of γ-OHPdG (FIG. 13A). Then, samples from forty-five HCC patients were used. Consistent with the results from mice, levels of γ-OHPdG in HCC did not correlate with those of the adjacent normal tissues (FIG. 13B).

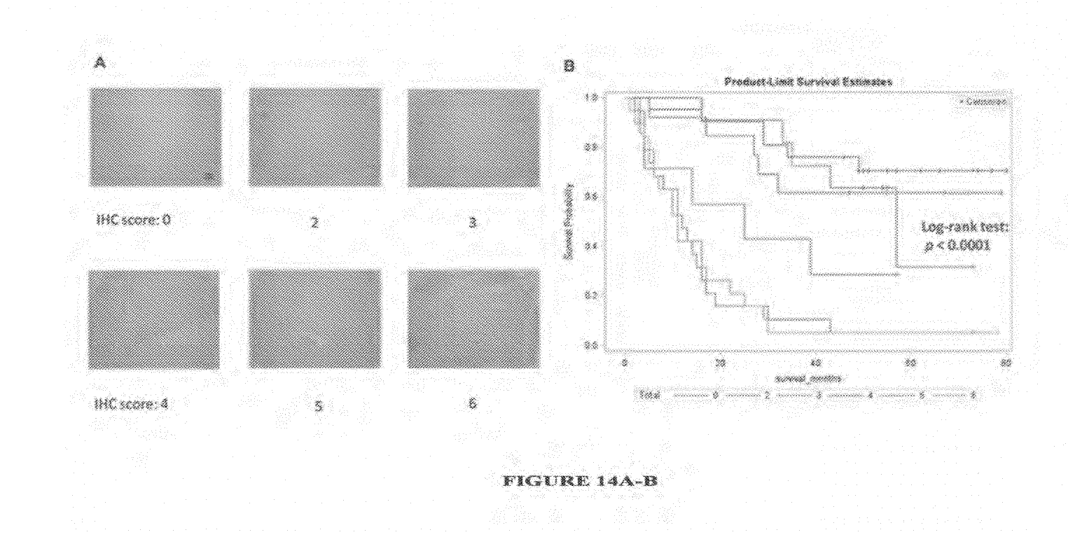

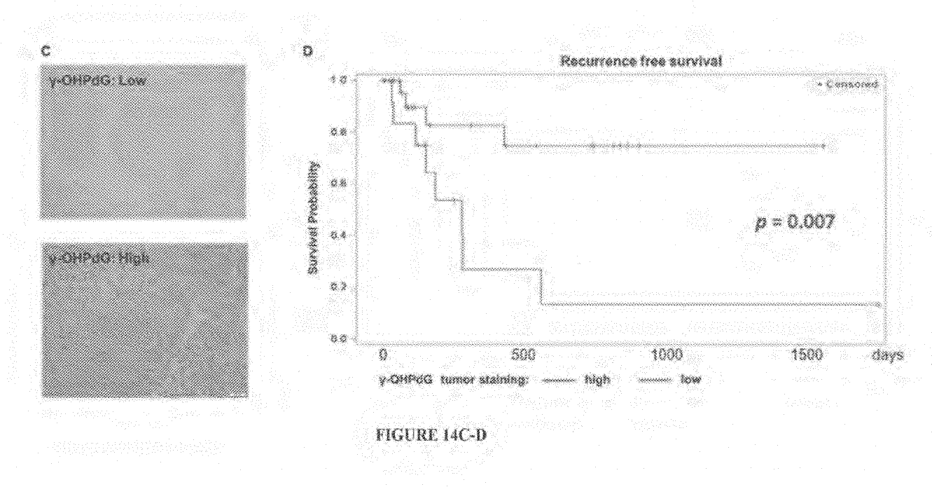

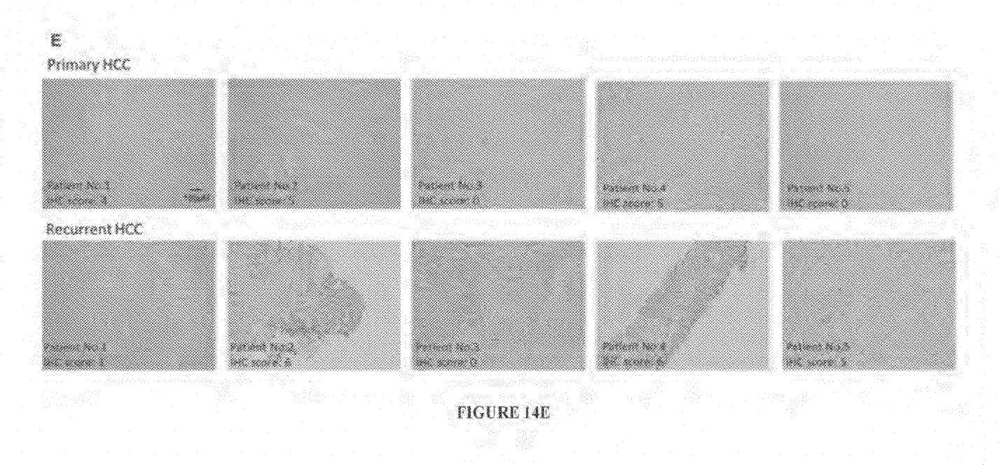

To determine whether γ-OHPdG serves as a predictive biomarker for the survival of HCC patients, liver samples were identified from a set of ninety HCC patients who underwent surgery without adjuvant therapy between August 2006 and November 2009. These patients were followed up for 4-7 years. It was found that the high γ-OHPdG levels in the tumor were strongly associated (p<0.0001) with poorer survival in these patients (FIGS. 14A and B). 45 patients were recruited and γ-OHPdG was examined with patient's recurrence-free survival. Patients with low γ-OHPdG tumors (IHC score <3) experienced a significantly prolonged HCC recurrence-free survival compared to patients with high γ-OHPdG tumors (IHC sore >3) (p=0.007, FIGS. 14C and D). After 2 years of follow-up from the date of curative surgical resection, the probability of no cancer recurrence in patients with low γ-OHPdG tumors is 75%, while the probability of no cancer recurrence in patients with high γ-OHPdG tumors is only 13% (p=0.0002, Table 4). These clinical studies support γ-OHPdG as a biomarker for predicting the risk of human HCC recurrence. Levels of γ-OHPdG in the recurrent tumors were compared to that in the primary HCC, and it was found that 80% of them showed similar levels of γ-OHPdG compared to their primary HCC tumors (FIG. 14E).

TABLE 4
Summary of probabilities of RFS (recurrence-
free survival) for γ-OHPdG in 45 HCC patients

| | Half a year | 1 year | 1 and half years | 2 years |
|---|---|---|---|---|
| Tumor γ-OHPdG | 0.83 | 0.83 | 0.75 | 0.75 |
| score ≤ 3 | | | | |
| Tumor γ-OHPdG | 0.64 | 0.27 | 0.27 | 0.13 |
| score > 3 | | | | |
| P value | 0.271 | 0.002 | 0.013 | 0.0002 |
| Normal γ-OHPdG | 0.75 | 0.62 | 0.54 | 0.54 |
| score ≤ 3 | | | | |
| Normal γ-OHPdG | 0.76 | 0.61 | 0.61 | 0.46 |
| score > 3 | | | | |
| P value | 0.956 | 0.963 | 0.754 | 0.727 |

Liver cancer is a type of cancer that evolves over the course of several decades and is extremely difficult to treat once diagnosed. Most patients with advanced disease after diagnosis only have a remaining lifespan of four-six months. Therefore, developing a mechanism-based biomarker is extremely important for liver cancer intervention. This work provides the first evidence that γ-OHPdG is repaired by NER in animals. Its levels are significantly higher in Xpa^{-/-} mice than WT mice. It was also found that γ-OHPdG levels increased age-dependently in both Xpa^{-/-} and WT mice, indicating the endogenous lesion accumulates in tissues over time. A similar trend was observed in LEC rats (an irregular copper accumulation HCC model) and the DEN-injected mice (an inflammation-related model).

In Xpa^{-/-} mouse liver tumors, the GC>TA mutation was overwhelmingly represented. Human HCC also have over-representation of GC>TA mutation, distinctly different from other human solid tumor mutation spectra. The mutation signature in this model coincides with the signature of mutational process of human HCC with a prevalence at 12.1%, implicating the role of DNA lesions on G:C pair in HCC. A relatively high frequency of deleterious mutation is also found. The mutation frequency observed in this study is completely different from that of skin tumor of these mice induced by UVB irradiation, in which a high frequency of C>T and CC>TT transitions at dipyrimidine sites (93%) on p53 mutation is found, which are characteristic of mutations caused by pyrimidine dimers. When comparing the mutations in human HCC, it was found that a number of genes overlapped, for example, up to 121 mutations per nodule are detected with a high frequency mutations on G:C pair. The explanation was primarily based on the methylation of cytosine. It has been known that methylation at CpG sites enhances γ-OHPdG formation at these sites; moreover, CpG methylation greatly increases γ-OHPdG-induced GC>AT and GC>TA mutation frequency. These data implicate γ-OHPdG as a potential cause of the high frequency mutations on CpG sites in the human HCC studies.

It was reasoned that, if the formation of DNA lesions which cause critical mutations can be inhibited, the carcinogenesis process may be efficiently attenuated. Theaphenon E showed the most potent effects in lowering γ-OHPdG, and it almost completely blocked liver cancer in Xpa^{-/-} mice and the DEN model. Antioxidant significantly prevents the carcinogen-induced liver carcinogenesis governed by hepatocyte Ikβ kinase that involves inflammation and elevated ROS. It was consistently observed that γ-OHPdG levels are higher in the livers of animals susceptible to liver carcinogenesis, and the decrease of its levels by antioxidants correlates well with inhibition of HCC formation. These results support γ-OHPdG as a risk biomarker of hepatocarcinogenesis. Importantly, this notion was further demonstrated using liver tumor specimens from HCC patients.

Oxidative stress has emerged as a crucial factor in the initiation and progression of HCC under various pathological conditions. It is known to be involved in migration, invasion, and metastasis of cancer, including HCC. Because cancer is a genetic disease, γ-OHPdG as a specific promutagenic DNA lesion associated with liver cancer is considered a more direct and mechanistically relevant biomarker for HCC development. These studies show that γ-OHPdG has potential as a prognostic tool in HCC recurrence and its prevention.

### Example II

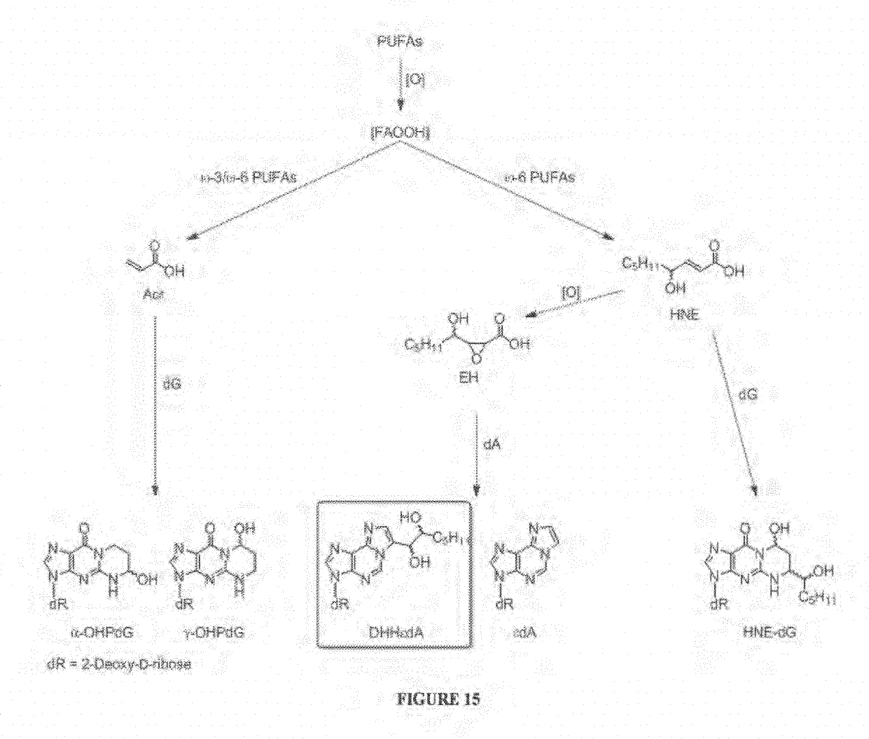

In addition to exposure to environmental carcinogens, endogenously formed DNA-reactive compounds could also play an important role in carcinogenesis. Lipid peroxidation (LPO) of polyunsaturated fatty acids (ω-3 and ω-6 PUFAs) is an endogenous source of protein and DNA damage. The oxidation of PUFAs produces α,β-unsaturated aldehydes (enals), such as (E)-4-hydroxy-2-nonenal (HNE) and acrolein (Acr), that can modify DNA bases forming cyclic DNA adducts (FIG. 15). HNE is a unique oxidation product of ω-6 PUFAs, such as arachidonic and linoleic acids. It is believed to be a major cytotoxic product of LPO. HNE reacts with deoxyguanosine (dG) via Michael addition to form cyclic 1,N² propanodeoxyguanosine adducts (HNE-dG). HNE-dG is mutagenic, however, its levels in vivo are often too low to be quantitatively and consistently detected by available t methods, such as ³²P-postlabeling and LC-MS/MS.

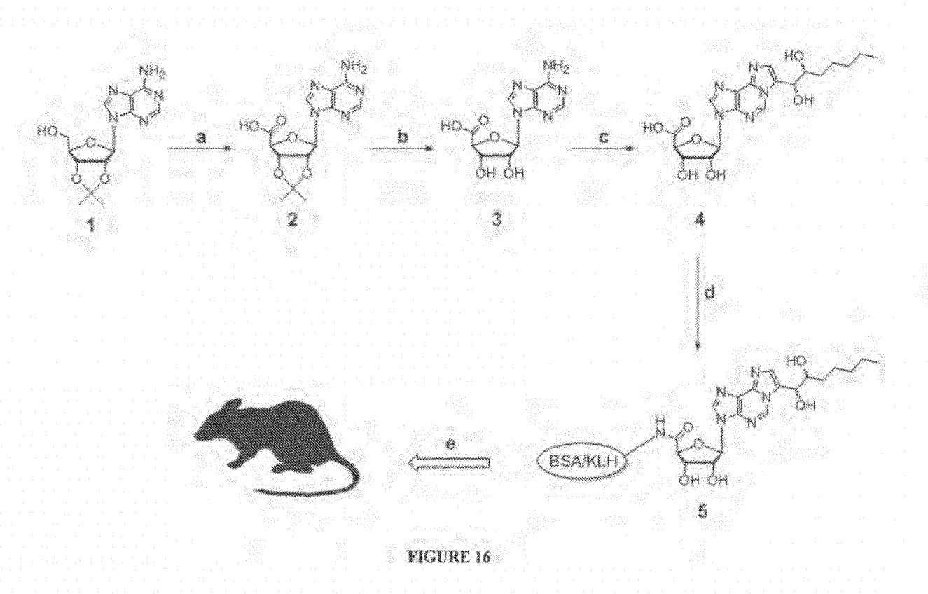

HNE is readily epoxidized to 3,4-epoxy-4-hydroxynonenal (EH) by hydrogen peroxide, organic peroxides, fatty acid hydroperoxide, and possibly lipoxygenase. Compared to HNE, EH is more reactive towards nucleobases. EH primarily reacts with deoxyadenosine (dA) and dG forming unsubstituted and substituted etheno adducts, such as 1,N⁶-ethenodeoxyadenosine (EdA), 1,N²-ethenodeoxyguanosine (1,N²-edG), 7-(1′,2′-dihydroxyheptyl)-1,N²-ethenodeoxyguanosine (DHHεdG) and 7-(1′,2′-dihydroxyheptyl)-1,N⁶-ethenodeoxyadenosine (DHHεdA) (FIG. 16). Unlike edA and 1,N²-edG, DHHEdA has only been detected as common background lesion in vivo recently. To investigate the role of DHHεdA in cancer promotion by ω-6 PUFAs, with respect to its formation, mutagenicity, repair, and/or interactions with proteins in signaling pathways in cells and tissues, specific monoclonal antibodies (mAbs) against DHHεdA were developed and characterized. The specificity and reactivity were determined by direct and competitive ELISA assays. The antibody was used to detect DHHεdA in DNA using a highly sensitive ELISA assay and in cell nuclei using flow cytometry-based fluorescence-activated cell sorting (FACS) analysis. The levels of DHHεdA in cellular DNA detected by the immunohistochemistry (IHC) methods were further confirmed by quantitative mass spectrometry.

### Chemicals

(E)-4-hydroxy-2-nonenal (HNE) and 2,3-epoxy-4-hydroxynonanal (EH) were provided by Dr. Shantu Amin of Penn State University. Synthesis of a DHHεdA standard and a [¹⁵N₅]-DHHεdA internal standard used for quantitative mass spectrometry was described in Fu et al., “In vivo detection of a novel endogenous etheno-DNA adduct derived from arachidonic acid and the effects of antioxidants on its formation,” Free Radic. Biol. Med. 73, 12-20 (2014). Water used in all experiments was from an in-house Milli-Q Biocel water polisher (EMD Millipore, Billerica, Mass.). All other chemicals, unless stated otherwise, were purchased from Sigma-Aldrich (St. Louis, Mo.) and were analytical or HPLC grade.

### Synthesis of 2′,3′-O-isopropylidene-5′-Carboxyadenosine (2)

2′,3′-O-isopropylideneadenosine (5.00 g, 16.27 mmol), 2,2,6,6-tetramethyl-1-piperidinyloxy free radical (TEMPO) (510 mg, 3.26 mmol) and (diacetoxyiodo)benzene (DIB) (11.53 g, 35.80 mmol) were placed in a 100 mL round bottom flask equipped with a magnetic stir bar. 30 mL of 1:1 acetonitrile (ACN):water was added and the mixture was stirred for 3 h. After a few initial minutes of stirring, the reaction mixture turns an orange-brown color and compounds begin to dissolve. Shortly after this, a white precipitate of compound 2 formed (FIG. 16). After 3 h the product was filtered, triturated by acetone (3×15 mL) and diethyl ether (3×15 mL) and dried overnight by vacuum. No further purification was necessary. The purity of compound 2 was analyzed by HPLC system 1 and was greater than 98%. The reaction yield was 91%. High-resolution mass spectrometry: expected mass 322.1151 m/z, observed mass: 322.1149 m/z.

### Synthesis of 5′-Carboxyadenosine (3)

The isopropylidene protecting group from compound 2 was removed by reacting 1.013 g (3.16 mmol) of compound 2 with 50 mL of 4:1 mixture of formic acid and water for 43 h at room temperature. The reaction progress was monitored using HPLC system 1. After completion of the reaction, the reaction mixture was evaporated using a centrifugal vacuum evaporator (SpeedVac (Waltham, Mass.)). Dry material was re-dissolved in dimethyl sulfoxide (DMSO) then purified using HPLC system 2. The purity of final material was greater than 98%. Reaction yield was 78%. High-resolution mass spectrometry: expected mass: 282.0838 m/z, observed mass: 282.0836 m/z.

### Synthesis of the 5′-carboxy derivative of 7-(1′, 2′-Dihydroxyheptyl) adenosine (Compound 4)

HNE (469 mg, 3.00 mmol) was dissolved in 1.2 mL of tetrahydrofuran (THF), 1.6 mL ACN and 3.25 mL (30 mmol) 30% hydrogen peroxide. The resulting mixture was added to compound 3 (527 mg, 1.88 mmol) dissolved in 50 mL of 2:1 THF:100 mM phosphate buffer (pH 7.4). The reaction was kept in a water bath at 50° C. for 7 days. 1 mL of 30% H₂O₂ was added after 19 h. The pH was checked periodically and adjusted by 1.0 M NaOH to pH 7.4. The reaction progress was followed using HPLC system 1. The excess of peroxides was removed using a saturated solution of sodium metabisulfite in water. The reaction mixture separated into two layers. Both layers were collected, evaporated on centrifugal vacuum evaporator, re-dissolved in water and purified using HPLC system 2. The purity of the final product was greater than 98%. Reaction yield was 24%. High-resolution mass spectrometry: expected mass, 436.1832 m/z, observed mass: 436.1851 m/z.

### Conjugation of Hapten 4 to Bovine Serum Albumin (BSA)

BSA (2 mg) was dissolved in 200 μL of 0.1 M 2-(N-morpholino)ethanesulfonic acid (MES), pH 4.7 buffer and mixed with compound 4 (0.5 mg, 0.0011 mmol) dissolved in 500 L of MES buffer. 1-(3-(dimethylamino)propyl)-3-ethylcarbodiimide hydrochloride (EDAC) (22 mg, 0.11 mmol) was dissolved in 1 mL of water and 100 μL (×10, 0.011 mmol) or 20 μL (×2, 0.0022 mmol) was added to the reaction mixture. The reaction mixture was stirred for 3 h at room temperature then then separated on 7K MWCO Zeba Spin Desalting columns (Thermo Scientific #89891, Fisher Scientific, Pittsburgh, Pa.) using water as an eluent. Protein concentration in the final eluent was determined by spectrophotometry. The conjugation was confirmed with MALDI-TOF/TOF mass spectrometry. Briefly 1 μL of protein with conjugated hapten was mixed with 1 μL of 10 mg/mL sinapinic acid in 30% (v/v) ACN containing 0.3% (v/v) trifluoroacidic acid (TFA) in water and then spotted on a MALDI plate. Mass spectra were acquired in linear high mass mode using a 4800 MALDI-TOF/TOF mass spectrometer (AB SCIEX, Framingham, Mass.).

Conjugation of hapten 4 to keyhole limpet hemocyanin (KLH)

Lyophilized Imject mcKLH in MES buffer (6 mg) (Thermo Scientific #77653, Fisher Scientific, Pittsburgh, Pa.) was reconstituted in 600 μL of water and mixed with compound 4 (5.4 mg, 0.012 mmol) dissolved in 1.5 mL of 0.1 M MES, 0.9 M NaCl, pH=4.7 buffer. A 150 μl (7.5 μmol) aliquot of EDAC (10 mg, 0.05 mmol) in water (1 mL) was added to solution of KLH containing compound 4. The reaction mixture was gently stirred for 2 h at room temperature and then separated on 7K MWCO Zeba Spin Desalting Columns using Imject Purification Buffer Salts as eluent (Thermo Scientific #77159, Fisher Scientific, Pittsburgh, Pa.). Protein concentration was confirmed as described above. The complex structure of KLH, its high molecular weight, and stability issues for this protein prohibited MALDI-MS analysis.

### Immunization

Immunization and antibody isolation/purification was done using a commercial service (GenScript USA Inc.; Piscataway, N.J.). Briefly, BALB/c mice were immunized with 100 μg of DHHεdA-conjugated to KLH in 0.1 mL of saline emulsified with an equal amount of Freund's complete adjuvant, given in a split dose, intraperitoneally (i.p.) and subcutaneously (s.c.). A second immunization was given 2 weeks after the first one in incomplete Freund's adjuvant. Mice were boosted with 100 μg of the conjugate in saline, administered i.p., on days 1, 2, 3 and 4 on the fourth week after the second injection. On day 5, mice were sacrificed and spleens were removed for fusion. Test bleeds were taken to check antibody reactivity towards immunogens using ELISA and DHHεdA-conjugated to BSA.

### Cell Fusion and Screening of Hybridomas

Satisfactory immune responses were seen in all mice, two of which were used for cell fusion and hybridoma production. A total of 20 hybridoma cell lines were produced. Splenocytes and myelomas were fused, plated into 96-well culture plates and screened by ELISA to detect the positive clones. Three selected clones were then subcloned by limiting dilution until they were monoclonal and stable hybridomas. Two subclones from each parental clone were produced expanded into culture flasks, and 4-6 vials of cells for each subclonal cell line were cryopreserved.

mAbs Isolation and Purification

mAbs were produced using a roller bottle cell culture technique. Briefly, selected hybridoma cells were cultured with Dulbecco's modified eagle medium (DMEM) containing 10% fetal bovine serum (FBS) at 37° C., 6% CO₂ in humidified incubator. When hybridoma cells were at >70% confluence, they were transferred to a T-25 tissue culture flask. After reaching >70% confluency, cells were suspended in roller bottle cell culture medium to make a concentration of 2.5-3.5×10⁴ cells/mL, and transferred to a tissue culture roller bottle in an electro-thermal incubator for about 10-14 days. Supernatant was collected when the cell density was below 1×10⁵ cells/mL. Supernatant was centrifuged, filtered with 0.22 μm filter, and concentrated using a centrifugal evaporator to obtain a final volume of 100 mL. mAbs were purified by affinity column chromatography using recombinant protein A resin (GenScript USA Inc.; Piscataway, N.J.).

Characterization of mAbs-ELISA and Competitive ELISA

The ELISA plate wells (white 96-well Nunc MaxiSorp, Thermo Scientific #436110, Fisher Scientific, Pittsburgh, Pa.) were coated with 100 μL of 0.1 μg/mL antigens of DHHεdA conjugated to BSA in PBS, at 37° C. for 2 h, and washed trice with PBS containing 0.1% (v/v) Tween 20 (PBST). The wells were then blocked with 100 μL of 1% (m/v) BSA in PBS at room temperature with moderate shaking for 1 h and washed trice with PBST. For ELISA, 100 μL of antibody solutions with different dilutions of antibodies or test bleeds in PBS containing 1% (w/v) BSA were added. For competitive ELISA, first the tested competing agents in 1% (w/v) BSA were added, then antibody solutions or test bleeds in PBS containing 1% (w/v) BSA were added into the wells. The plates were incubated at 37° C. for 1 h and washed trice with PBST. Horseradish peroxidase conjugated goat anti-mouse IgG secondary antibodies in 100 μL of PBS containing 1% (w/v) BSA were added, and the plates were incubated at 37° C. for 30 min. The wells were washed twice with PBST and once with PBS and developed with 100 μL of working solution of SuperSignal ELISA Femto Maximimum Sensitivity Substrate kit (Thermo Scientific #37074, Fisher Scientific, Pittsburgh, Pa.). Protected from light plates they were gently shaked for 3-4 min at room temperature. Then, luminescence intensity was measured on plate reader (GloMax-Multi Detection System, Promega, Madison, Wis.).

Characterization of mAbs-Detection of DHHεdA in EH Modified Calf Thymus DNA Samples by ELISA

The ELISA plate wells were coated with 50 μL of EH modified calf thymus DNA (CT DNA) (see below) in PBS (20 μg/mL to 2 ng/mL). Then, buffer was evaporated overnight at room temperature. Afterwards, evaporation plates were washed five times with 100 μl PBST. The wells were then blocked with 200 μL of 1% (m/v) BSA in PBS at room temperature with moderate shaking for 1 h and washed trice with 200 μL PBST. Purified antibody solution (50 μl, 1:200,000 dilution) in 1% (w/v) BSA were added and the plates were incubated at 37° C. for 1 h and washed trice with PBST. Horseradish peroxidase conjugated goat anti-mouse IgG secondary antibodies in 50 μL of PBS containing 1% (w/v) BSA were added, and the plates were incubated at 37° C. for 30 min. The wells were washed twice with PBST, once with PBS and then developed with 50 μL of working solution of SuperSignal ELISA Femto Maximimum Sensitivity Substrate kit (ThermoFisher). Protected from light plates the wells were gently shaken for 3-4 min at room temperature. Then, luminescence intensity was measured on a plate reader.

### Cell Culture and Treatments

HepG2 cells (#HB-8065, ATCC, Manassas, Va.) were grown in complete DMEM (#10-013, Corning Life Sciences, Tewksbury, Mass.) until 85-90% confluent. Cells were treated with a final concentration of 100 μM or 300 μM of either arachidonic acid (AA) or EH solubilized in 70% EtOH for 24 h. Cells were then scraped down in media and transferred to a centrifuge tube and spun for 3000 rpm for 5 min. The pellet was washed 2× with 1×PBS, centrifuging between washes. DNA was then isolated from the cells and prepared for LC-MS/MS-MRM, after enzymatic hydrolysis as described above.

Primary human hepatocytes (#HUFS1M, Lot#HUM4132, Triangle Research Labs, Durham, N.C.) were cultured in hepatocyte media (#5201, ScienCell Research Laboratories, San Diego, Calif.) until 85-90% confluent. Cells were then treated with a final concentration of 300 μM EH dissolved in 70% EtOH, added to the culture media. The cells were collected at the designated time points following EH treatment: 0, 4, 8, 12 and 24 h. The nuclei were extracted using Nuclei EZ Prep Nuclei Isolation Kit (#NUC-101, Sigma-Aldrich, St. Louis, Mo.) and fixed with 3.7% formaldehyde for 10 min. The nuclei were then prepared for FACS analysis using the method shown previously.

### FACS Analysis

The geometric mean of the fluorescence intensity of Alexa Fluor 488-labeled DHHεdA positive cells was measured using FACS. The output values of triplicate control and EH treatment groups (see above) were averaged and the standard deviation was calculated using Excel. Additionally, a Student's t-test was performed to determine significance by comparing the triplicate values for the control group to those of the EH treated group for each individual time point of 4, 8, 12, and 24 h.

### EH-Modified Calf Thymus (CT) DNA

EH was generated by reacting HNE (20.6 mg) with 30% hydrogen peroxide (21.4 μL) in a mixture of 250 μL THF, 10 μl ACN and 8 mg sodium carbonate over 1 h at room temperature. At the end of the reaction, 1 mL of chloroform and 1 mL of water were added, and after vigorous shaking. Chloroform fraction was collected, washed twice with 1 mL of water, dried over anhydrous sodium sulfate, filtered, and evaporated using vacuum centrifugal evaporator. After evaporation, the reaction mixture was dissolved in 100 μL of ethanol (EtOH) and used directly to modify DNA.

CT DNA (0.5 mg in 0.5 mL phosphate buffered saline pH 7.4 (PBS)) was mixed with L of previously prepared EH solution and kept at 37° C. for 19 h. The solution was extracted three times by 0.5 mL of chloroform, then DNA was precipitated by adding 100 μL of cold 4M sodium chloride solution and 1.5 mL of cold EtOH. The sample was kept for 30 min at −20° C., centrifuged, washed twice by cold 80% EtOH and air dried. DHHεdA modification levels were checked by mass spectrometry.

### DNA Isolation and Hydrolysis for LC-MS/MS-MRM

DNA was isolated by a QIAGEN Blood and Cell Culture DNA Maxi Kit (#13362, QIAGEN Inc., Valencia, Calif.) using the protocol recommended by the manufacturer. For hydrolysis, dry DNA (50 to 1000 μg) was dissolved in 5 mM magnesium chloride and 0.5 mM GSH solution (1 mL per 1 mg of DNA), then 50 fmol of [¹⁵N₅]-DHHεdA 1,2 and 50 fmol of [¹⁵N₅]-DHHεdA 3,4 were added as internal standards. DNA was hydrolyzed by incubation with DNase I (1300 units per mg of DNA, #D4527-40KU, Sigma-Aldrich, St. Louis, Mo.) for 30 min at 37° C., followed by a second addition of DNase I (1300 units per mg of DNA) and incubation for an additional 10 min at 37° C. Finally, phosphodiesterase I (0.06 units per mg DNA, #P3243-1VL, Sigma-Aldrich, St. Louis, Mo.), alkaline phosphatase (380 units per mg DNA, #10 108 146 001, Roche Diagnostic GmbH, Mannheim, Germany) and adenosine deaminase (0.5 units, #LS009043, Worthington Biochemical Corporation, Lakewood, N.J.) were added and the sample was incubated for 60 min at 37° C. After hydrolysis, a small portion of the hydrolysate was saved for further T quantification, and the remaining sample was purified using Phenomenex Strata-X 33μ 30 mg/1 mL polymeric reverse phase solid phase extraction columns (#8B-S100-TAK, Phenomenex, Torrance, Calif.). Before loading, samples columns were washed by ACN (3×1 mL) and stabilized by 25 mM ammonium formate pH=4.00 (3×1 mL). After loading, DNA hydrolysate columns were washed by 2.5% ACN in 25 mM ammonium formate pH=4.00 (1×1 mL), 5% ACN in 25 mM ammonium formate pH=4.00 (1×1 mL), followed by DHHεdA collection by 30% ACN in 25 mM ammonium formate pH=4.00 (1×1 mL). The DHHεdA fraction was dried using a SpeedVac rotary concentrator, re-dissolved in 400 μL 1:1 water:ACN, transferred to HPLC vials, dried and kept at −20° C. Before quantification, samples were dissolved in 60 μL of water and 37 μL was injected on LC-MS.

### Quantification of DHHεdA in DNA by LC-MS/MS-MRM

Quantification was carried out on a SCIEX 6500 QTRAP triple quadrupole mass spectrometer (AB Sciex LLC, Framingham, Mass.) interfaced with a Waters ACQUITY UPLC liquid chromatography system equipped with Waters ACQUITY UPLC BEH C18 50×2.1 mm, 1.7 μm particle size column (Waters Corporation, Milford, Mass.). The separation of adducts was performed isocratically by eluting with 13.5% ACN, 1 mM ammonium formate buffer over 6.5 min using 0.5 mL/min flow rate at 40° C., followed by 100% ACN wash. The ESI source operated in positive mode. The MRM experiment was performed using ion transitions of 406.2-290.2 m/z (DHHεdA) and 411.2-295.1 m/z ([¹⁵N₅]-DHHεdA) with CE of 28 eV for quantification, and those of 406.2-160.1 m/z (DHHεdA) and 411.2-165.0 m/z ([¹⁵N₅]-DHHεdA) with a CE of 76 eV were used for structural confirmation. All other parameters were optimized to achieve maximum signal intensity. Calibration curves were constructed for two HPLC resolved peaks before each analysis using standard solutions of DHHεdA and [¹⁵N₅]-DHHεdA. A constant concentration of [¹⁵N₅]-DHHεdA (1 fmol/μL) was used with different concentrations of DHHεdA (3.3 amol/μL-65 fmol/μL) and analyzed using 37 μL injections by LC-MS/MS-MRM. The standard curves were linear in the range from 0.37 to 800 fmol of DHHεdA on column for both peaks (1/x weighting; r²=0.9998 and r²=0.9984 for both LC resolved pairs of DHHεdA stereoisomers 1,2 and 3,4 respectively). The measured limit of quantification (LOQ) was 0.37 fmol/column and the limit of detection (LOD) was 0.1 fmol/column for both peaks. The overall method detection limit (MDL) for DNA samples was calculated to be 1-5 fmol of each HPLC resolved isomers per sample. To express adduct levels as number of adducts per unmodified bases T was quantified in DNA hydrolysate using HPLC System 3 with detection at 258 nm. A standard curve (from 5 nmol to 5 pmol of T on column) was constructed using UV quantified T standard (ε₂₆₇=9650 M⁻¹×cm⁻¹ in water).

### High Resolution Mass Spectrometry

High resolution mass spectrometry was performed in the Metabolomics and Proteomics Shared Resources of the Lombardi Comprehensive Cancer Center (Georgetown University, Washington D.C.) using a Waters ESI-Q-TOF Premiere mass spectrometer with Waters ACQUITY UPLC as the front end (Waters Corporation, Milford, Mass.).

### HPLC Systems

System 1 was an Agilent 1200 HPLC system that included a G1322A degasser, a G1311A quaternary pump, and a G1315D photodiode array detector (Agilent Technologies, Inc., Santa Clara, Calif.). The system was equipped with a Phenomenex Prodigy ODS3, 250×4.6 mm, 100 Å, 5 m column protected by a Phenomenex guard cartridge (Phenomenex, Inc., Torrance, Calif.). Solvent A was water with 0.1% TFA; solvent B was ACN with 0.1% TFA. A flow rate of 1 mL/min was established. The gradient program was: 0-25 min from 0% B to 100% B, followed by 12 min washing by 100% B. Before each run, the column was equilibrated with 100% A for 12 min. The detector was recording chromatograms at 200, 210, 227, 254 and 280 nm. Spectra for all time-points were recorded from 190 to 400 nm with a resolution of 1 nm.

System 2 was a Shimadzu HPLC system comprised of a SPD-M10A VP diode array detector, a SCL-10A VP controller and two LC-10AD VP pumps (Shimadzu Scientific Instruments, Columbia, Md.), equipped with Phenomenex Prodigy 250×21.2 mm, 5 m particle size, 100 Å, ODS3 (C18) columns (Phenomenex, Torrance, Calif.). Solvent A was water with 0.1% TFA; solvent B: ACN with 0.1% TFA. Flow rate was set to 9.999 mL/min. The gradient program was: 0-6 min 100% A, 6-96 min from 0% B to 100% B followed by 20 min washing with 100% B. Before each run the column was equilibrated with 100% A for 20 min. The detector was recording chromatograms from 200 to 320 nm with 1 nm resolution and 4.2 Hz sampling rate.

System 3 was a Waters ACQUITY UPLC system with PDA detector and Waters ACQUITY UPLC BEH C18 50×2.1 mm, 1.7 m particle size column (Waters Corporation, Milford, Mass.). Solvent A was water with 0.1% heptafluorobutyric acid (HFBA), solvent B was methanol (MeOH) with 0.1% HFBA. Column temperature was 40° C. and the flow rate was 0.5 mL/min with linear gradient program: 0 to 4 min from 0 to 4% B. Between injections column was washed by 100% B for 2.6 min and stabilized with 100% A for 2.6 min.

Developing mAbs Against DHHεdA

The vicinal diols in the sidechain of DHHεdA hapten made its conjugation to carrier proteins challenging as the standard periodate oxidation strategy will result in sidechain cleavage. An alternative conjugation strategy, involving conversion of guanosine 5′-hydroxy group into 5′-carboxy and conjugation of modified hapten to protein by water soluble carbodiimide (FIG. 16), was used. Mass spectrometry confirmed that the molecular weight represents the modification levels of 3.7 (×2) and 10.0 (×10) adducts per BSA. Levels of modifications for KLH conjugate were not confirmed by mass spectrometry due to high molecular mass, complex structure and poor stability of carrier protein.

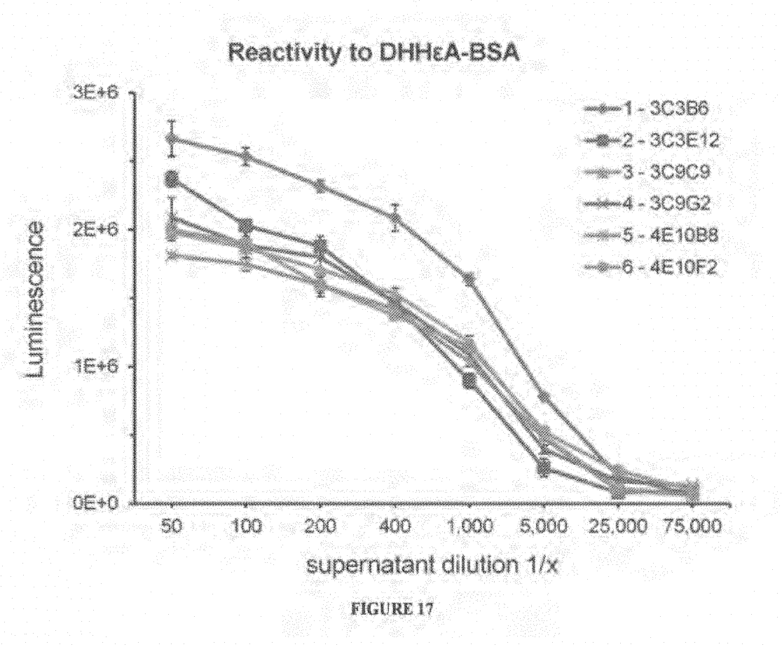

Five mice were immunized with DHHεA-KLH and all of them showed satisfactory immune responses. Based on ELISA specificity and selectivity results, two mice were chosen for further antibody development. After spleen fusion a total of 20 hybridoma cells were produced. The supernatants of all fused cells lines were screened by ELISA for their activity towards DHHεdA from which three positive clones were chosen. They were cloned into two subclones each to form stable monoclonal hybridomas (3C3B6, 3C3E12, 3C9C9, 3C9G2, 4E10B8, 4E10F2). They were further tested by direct and competitive ELISA for their activity and specificity against antigen. The supernatants from the selected cell lines were used without further purification. For all competitive ELISA experiments mAbs concentrations were normalized by total protein content in the cell supernatants. To test activity sequential dilutions of mAbs were incubated with 100 ng of DHHεA-BSA conjugate (×2) deposited on ELISA plate. mAbs from all six anti DHHcdA monoclonal cell lines displayed similar and strong binding activities (FIG. 17).

### Determining Reactivity and Specificity of Antibodies

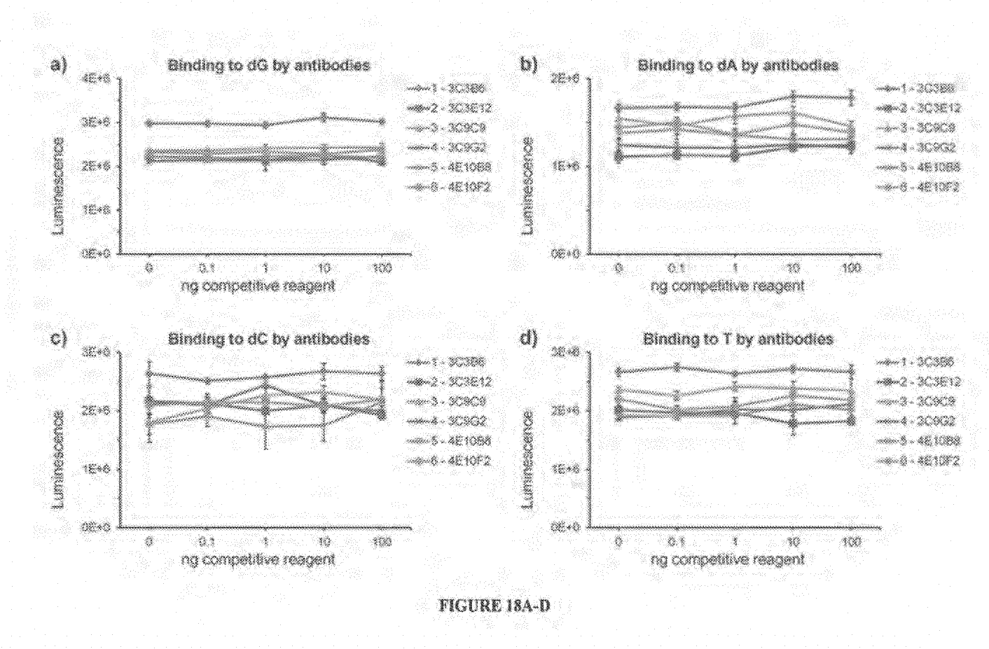

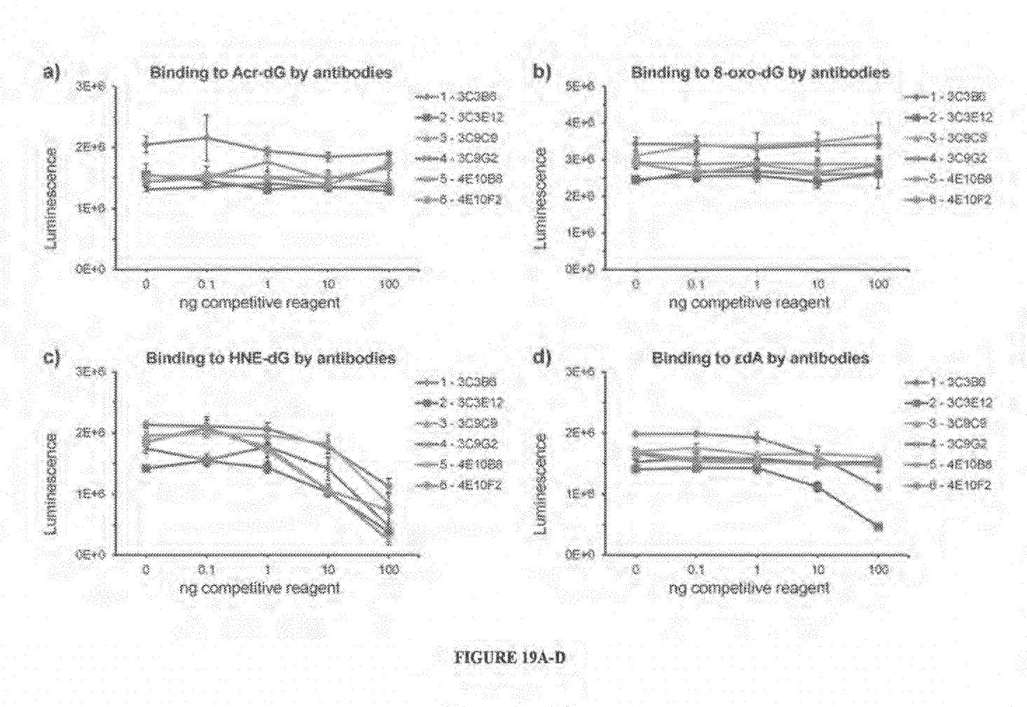

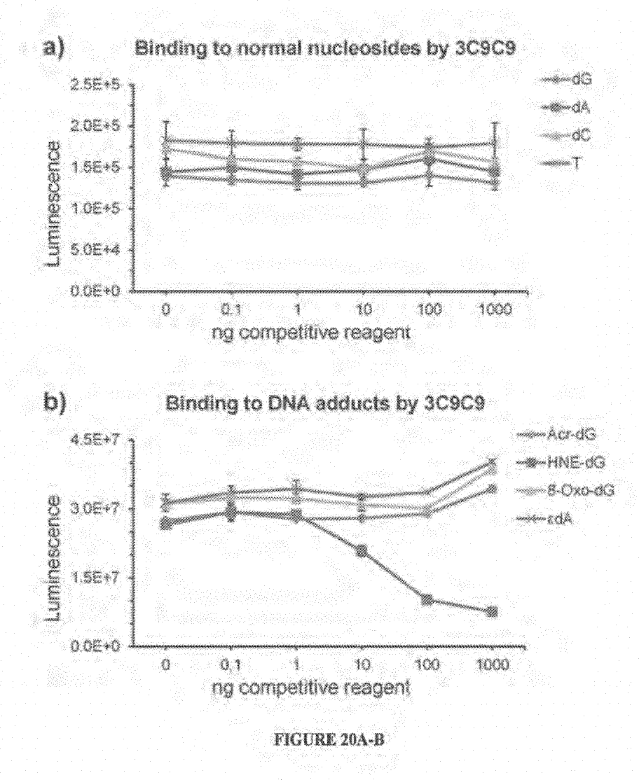

The specificity of mAbs was determined using competitive ELISA by testing reactivity towards immunogen in the presence of normal nucleotides and selected cyclic DNA adducts. All six mAbs showed no reactivity towards normal nucleotides, including dA (FIG. 18). Also, no reactivity towards Acr-dG or 8-oxo-dG was observed for all mAbs (FIG. 19A, 19B). All six developed mAbs showed weak reactivity towards HNE-dG only at concentrations above 10 ng/well (FIG. 19C). The competing effects of HNE-dG at 100 ng/well was similar or weaker than competing effects for DHHεdA at one ng/well in that assay, indicating that the mAbs have at least hundred times stronger affinity towards DHHεdA than HNE-dG. Because of the relatively low levels of endogenous HNE-dG compared to DHHεdA in vivo, the weak cross-reactivity is not expected to affect the specificity of the mAbs to detect DHHεdA. The two cell lines showing moderate competitive effects against edA (FIG. 19D) were not further used in the study. All other mAbs showed similar affinity and specificity. However, because 3C9C9 performed slightly better in the competitive ELISA of DHHεdA vs. HNE-dG (FIG. 19C), 3C9C9 was used to produce purified mAb. 3C9C9 mAb was then evaluated against dA, dC, dG, T, Acr-dG, 8-oxo-dG and edA to confirm that it has no cross-reactivity and similar results were obtained (FIG. 20A,B). mAb reactivity towards 7-(1,2-dihydroxyheptyl)-edG (DHHεdG) was not tested because, when this adduct forms, it is rapid converted to 1,N²-edG under neutral and basic conditions.

### Detection of DHHεdA in DNA

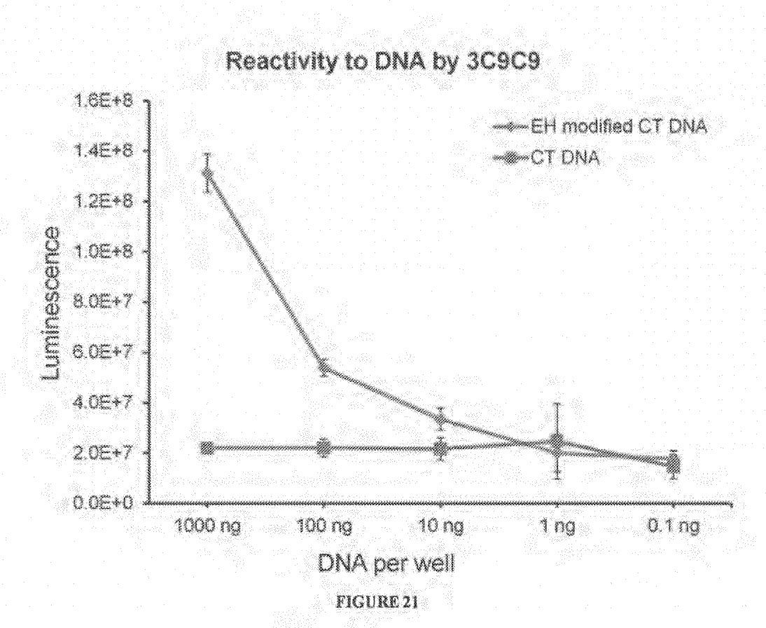

The antibody was used to determine the sensitivity for detecting DHHεdA in DNA. EH-modified CT DNA was used. The levels of modification were determined by quantitative mass spectrometry to be 469.8 adducts per 10⁶ unmodified nucleotides (in the absence of dA in DNA hydrolysate, T was quantified as dA complementary base) or 272.3 fmol of adduct per 1 μg of DNA. The endogenous DHHεdA levels detected in unmodified CT DNA were 21.3 adducts per 10⁹ unmodified nucleotides (12.3 amol adduct per 1 μg of DNA). EH modified CT DNA and CT DNA were coated on ELISA plates using amounts of DNA from 1 μg to 0.1 ng per well. The plates were incubated with 3C9C9 mAb then HRP-conjugated secondary antibody and finally ultrasensitive chemiluminescent HRP substrate was added to enhance signal. FIG. 21 shows the signals increase gradually with the amount of EH-modified DNA immobilized on plate, whereas the signals from unmodified CT DNA remain steady. The assay clearly showed that 3C9C9 mAb recognizes DHHεdA in DNA. The lowest amount of adduct detected in this assay was 2.7 fmol/well. The highest detectable amount was 272.3 fmol/well, as limited by a maximum coating of 1 μg of DNA/well. The absolute sensitivity in the ELISA assay was roughly seven times lower than the LC-MS/MS method (2.7 fmol of DHHεdA/well vs. 0.37 fmol/column). Practical sensitivity was also affected by maximum sample loading. For ELISA it was 1 μg of DNA/well, whereas in the MS assay, a few hundred g of DNA per injection can be used. An obvious advantage of ELISA is its ability to work directly with DNA without hydrolysis.

### Detection of DHHεdA in Human Hepatocytes

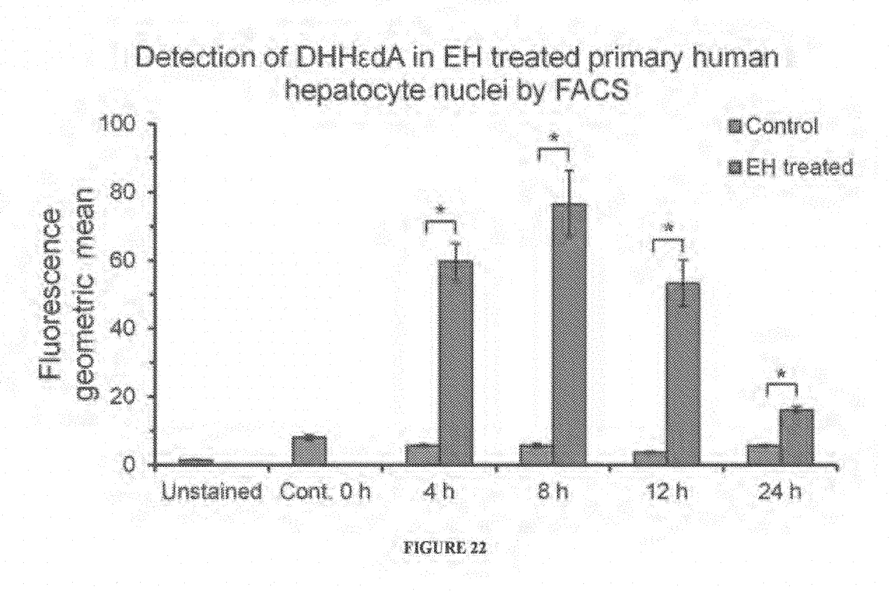

To quantify the formation of DHHεdA in human cells, HepG2 cells were treated with AA or EH for 24 h, using both a low dose (100 μM) and a high dose (300 μM). Upon autoxidation, AA generates HNE which can ultimately form EH via epoxidation. However, the AA treatment resulted in a detectable level only in the high dose. Both low and high dose treatments of EH induced formation of DHHεdA with 153.5 and 2230.4 adducts/T×10⁹, respectively. Formation of DHHεdA was determined using LC-MS/MS (Table 5). EH treatment was used in subsequent experiments, to determine the application of the purified 3C9C9 antibody in human cells. Primary human hepatocytes were treated with a final concentration of 300 μM EH. Human hepatocytes are a storage source for lipids and de novo lipid synthesis, and are sites of lipid peroxidation-induced cell injury. Primary human hepatocytes were used as they are not a modified cell line and may be more physiologically relevant than the HepG2 cells. Primary human hepatocytes were treated then harvested at 4, 8, 12 and 24 h. Extracted nuclei of treated cells were used to minimize the interference from cross-reactivity of 3C9C9 with components of the cytoplasm. FACS analysis showed a significant difference between control and EH treated cells for every time point across the 24 hour experiment (FIG. 22). A similar mean fluorescence intensity of DHHεdA was detected in EH treated cells at 4, 8, and 12 hours, however at 24 h, a decrease of DHHεdA was observed; possibly due to repair.

TABLE 5
DHHεdA levels found in HepG2 cellular
DNA after EH and AA treatment.

| | DHHεdA peak 1 | DHHεdA peak 2 | Total DHHεdA |
|---|---|---|---|
| Sample | [adducts/T × 10⁹] | [adducts/T × 10⁹] | [adducts/T × 10⁹] |
| CEH | bdl | bdl | bdl |
| LEH | 14.1 | 139.4 | 153.5 |
| HEH | 186.6 | 2043.8 | 2230.4 |
| CAA | bdl | bdl | bdl |
| LAA | bdl | bdl | bdl |
| HAA | 29.3 | 31.9 | 61.2 |

### Immunohistochemistry (IHC)

DHHεdA is promising as a biomarker of damage caused by oxidatized ω-6 PUFAs. As shown in the ELISA and FACS studies described herein, 3C9C9, an antibody that specifically binds to DHHεdA could be useful in diagnosing and treating liver cancer.

A number of embodiments have been described. Nevertheless, it will be understood that various modifications may be made. Accordingly, other embodiments are within the scope of the following claims.

What is claimed is:

1. A method of reducing the recurrence of liver cancer in a subject, comprising:
(a) obtaining a sample from a subject with liver cancer;
(b) treating the subject for liver cancer;
(c) determining a level of γ-hydroxy-1, N²-propanodeoxyguanosine (γ-OHPdG) in the sample;
(d) comparing the determined level of step c) to one or more control values, wherein an elevated level of γ-OHPdG indicates the subject is at risk for the recurrence of liver cancer; and
(e) administering an effective amount of an antioxidant to the subject.

2. The method of claim 1, wherein the step of obtaining the sample is performed prior to, concurrently with, or subsequent to treatment for liver cancer.

3. The method of claim 1, wherein the treatment is one or more of surgery, chemotherapy and radiation therapy.

4. The method of claim 1 wherein the antioxidant is Theaphenon E or α-lipoic acid.

5. The method of claim 1 wherein two or more antioxidants are administered to the subject.

6. (canceled)

7. (canceled)

8. (canceled)

9. (canceled)

10. (canceled)

11. A method of reducing the recurrence of liver cancer in a subject, comprising:
(a) obtaining a sample from a subject with liver cancer;
(b) treating the subject for liver cancer;
(c) determining a level of 7-(1′,2′-dihydroxyheptyl)-1,N⁶-ethenodeoxyadenosine (DHHεdA) in the sample;
(d) comparing the determined level of step c) to one or more control values, wherein an elevated level of DHHεdA indicates the subject is at risk for the recurrence of liver cancer; and
(e) administering an effective amount of an antioxidant to the subject.

12. The method of claim 11, wherein the step of obtaining the sample is performed prior to, concurrently with, or subsequent to treatment for liver cancer.

13. The method of claim 11, wherein the treatment is one or more of surgery, chemotherapy and radiation therapy.

14. The method of claim 11, wherein the antioxidant is Theaphenon E or α-lipoic acid.

15. The method of claim 11, wherein two or more antioxidants are administered to the subject.

16. (canceled)

17. (canceled)

18. (canceled)

19. (canceled)

20. (canceled)

21. A hybridoma cell line selected from the group consisting of hybridoma cell line hybridoma cell line 3C3B6, hybridoma cell line 3C3E12, hybridoma cell line 3C9C9, hybridoma cell line 3C9G2, hybridoma cell line 4E10B8, and hybridoma cell line 4E10F2.

22. The monoclonal antibody produced by the hybridoma cell line of claim 21.
